(19) United States
(12) Patent Application Publication
(10) Pub. No.: US 2011/0190623 A1
(43) Pub. Date: Aug. 4, 2011

(54) THERMALLY-ACTIVATABLE LIPOSOME COMPOSITIONS AND METHODS FOR IMAGING, DIAGNOSIS AND THERAPY

(75) Inventors: King Chuen Li, Houston, TX (US); Zheng Li, Houston, TX (US); Guoting Qin, Houston, TX (US)

(73) Assignee: The Methodist Hopsital Research Institute, Houston, TX (US)

(21) Appl. No.: 13/057,671

(22) PCT Filed: Aug. 5, 2009
(86) PCT No.: PCT/US09/52895

§ 371 Date: Apr. 19, 2011

Related U.S. Application Data
(60) Provisional application No. 61/086,392, filed on Aug. 5, 2008.

(51) Int. Cl. A61B 8/00; A61K 9/127; C12N 5/00; C12N 5/071; A61K 49/00; A61K 51/00; A61P 29/00; A61P 35/00; A61P 37/00; A61P 25/00; A61P 31/00; A61B 5/055; A61B 6/03; A61M 37/00

(52) U.S. Cl. 600/420; 424/450; 435/375; 435/366; 424/9.1; 424/1.21; 600/431; 600/425; 604/22; 604/20

(57) ABSTRACT

Disclosed are thermally-activatable liposomal compositions and methods for their use in the formulation and administration of therapeutic, prophylactic, and diagnostic agents. The disclosed liposome structures are capable of carrying a variety of biologically active reagents, and permitting their controlled release in vivo by exploiting properties of their thermoregulatable lability.

### FIELD OF THE INVENTION

The present invention relates generally to the fields of medicine and pharmaceuticals. More particularly, it concerns thermally activatable liposome formulations and methods for their use in the preparation and administration of one or more imaging agents, diagnostics, therapeutics, prophylactics, or pharmaceutical agents to an animal in need thereof.

### BACKGROUND OF THE INVENTION

### Description of Related Art

### Liposomes

Liposomes have been extensively developed and used during the past twenty-five years as drug delivery vehicles. The desired properties of efficient drug carriers include (a) the ability to evade the host's mononuclear phagocyte system to prolong the circulation half-life (t_{1/2}); (b) the preferential release of the encapsulated drug at the selected target site, and (c) an overall facility of routine administration.

While sterically stabilized liposomes have increased circulation t_{1/2} considerably, and fusogenic liposomes have facilitated limited cytoplasmic delivery of membrane-impermeable molecules, the creation of thermally activatable liposomes has trailed in development. The use of liposomes to carry diagnostic or therapeutic agents, drugs or other active agents either contained within the aqueous interior space (water soluble active agents) or partitioned into the lipid bilayer (water-insoluble active agents) has been extensively described in the art.

As noted in U.S. Pat. No. 6,726,925 to Needham, anti-neoplastic agents, and other drugs that have short half-lives in the bloodstream, are well suited to delivery via liposomes. Moreover, encapsulation of cytotoxic agents within liposomes typically helps to reduce their systemic toxicity.

Only a few approaches, however, have been reported in the literature for successfully producing temperature-sensitive liposomes that are useful as controlled-release diagnostic molecule or drug delivery vehicles. One such approach has focused on the phase-transition properties of constituent lipids (including, e.g., dipalmitoylphosphatidylcholine [DPPC]) which has a phase-transition temperature of 42.5° C.

### Heat-Sensitive Liposomes

Heat-sensitive liposomes have been used in research and clinical trials for delivery of therapeutic agents to target sites that can be enhanced by heating the target tissue. A main limitation with such compositions, however, is that the heat-sensitive liposomes used either require formulation with components that are not effective drug delivery vehicles, or are unstable at human body temperature, which leads to rapid (minutes to hours) release, often even in the targeted cell(s) or tissue(s) (see e.g., U.S. Pat. Nos. 5,094,854; 5,209,720; 5,720,976; 5,810,888; 6,200,598; 6,623,430; 6,690,976; 6,726,925; and 6,964,778).

Despite some developments as noted above, efficient temperature-controlled release from liposomes has been unpredictable and not yet been satisfactorily achieved for in vivo drug delivery applications. As such, there continues to be a need for more-effective drug delivery compositions and methods. Similarly, there exists a need for providing diagnostic and imaging reagents, in more localized and more-efficient methods than afforded by conventional systemic delivery; liposomal formulations according to the present invention are now believed to achieve these and other goals.

### SUMMARY OF THE INVENTION

The present invention provides new and useful compositions, as well as methods of employing them that may advantageously improve delivery of therapeutic, diagnostic and/or prophylactic agents to an animal in need thereof.

Embodiments of the present invention provide thermally-activatable, liposomes that include one or more polymerizable lipids, which combine the desirable properties of heat-sensitive liposomal delivery, with the stability afforded by polymerizable lipids that are able to remain at least substantially, and preferably entirely, intact for pre-determined periods after introduction into the body of a recipient animal. Such properties afford the medical arts an improved capability for more specifically controlling the release of the liposomes' contents at a selected time, and/or at a selected target site within the body of an animal to which the liposomal formulation has been administered or to which the liposomal formulation has been directed following administration.

In an overall and general sense, the formulations of the invention may include at least a first polymerized lipid and at least a first unpolymerized lipid. By controlling the ratio of polymerized to unpolymerized lipids in the drug-delivery vehicles of the invention, a range of partially-polymerized may be created that have unique properties with respect to thermal stability and delivery of one or more active ingredients, also referred to herein as pharmaceutical agent(s) entrapped within, or bound into releasable association with, the lipids.

The present invention provides liposomes that are sensitive to changes in the temperature of their surrounding environment. In one aspect, the thermosensitivity of such liposomes allows the release of a portion of one or more compounds or compositions entrapped within the interior aqueous space of the liposome, and/or the release of compounds associated within or about the lipid bilayer, at a target site that is either heated (e.g., in hyperthermia or thermotherapy) or that is at an intrinsically higher temperature than the rest of the body (e.g., in inflammation).

In embodiments of the invention, the presence of a non-polymerizable lipid component in the liposome causes it to become “leaky” at greater-than body temperature, and thereby release at least a first portion of the active ingredient(s) contained therein. These liposomes are particularly useful in the localized delivery of drugs or diagnostic agents, where the thermosensitive liposome is formulated to contain one or more compound(s) that can be delivered to a preselected target site in a patient's body. The target site may be either artificially heated (hyperthermia), or the target site may be at a higher temperature than non-targeted sites in the body due to natural causes (e.g., inflammation).

In particular embodiments, the invention provides thermosensitive liposomal formulations that release entrapped or encapsulated contents at temperatures that can be achieved in clinical settings using mild- to moderate localized hyperthermia. For the purposes of illustration, in various examples presented herein, the invention provides liposomes that are highly stable at body temperature (e.g., approximately 37° C.) but that become unstable and show enhanced release of entrapped compounds at temperatures at and above about 40° C.

The present invention provides compositions and methods for delivering diagnostic or therapeutic agents in a liposomal carrier, wherein the active agents are substantially released from the liposomes under mildly- to moderately-hyperthermic temperatures (i.e., about 40° C. to about 45° C. or so).

The thermally-activatable partially-polymerized liposome compositions disclosed herein are preferably formulated to be quite stable at one temperature (e.g., about normal human body temperature, i.e., about 37° C.), but quite unstable at mildy- to moderately-elevated temperatures (e.g., from about 39° C. to about 50° C.), as compared to that starting temperature. The ability of the liposomal formulations to “open” and “close” at different temperatures facilitates the development of unique diagnostic or treatment regimens wherein the temperature of the point of administration (e.g., the circulatory system) and the site of intended delivery in the selected tissue(s) and/or cell(s) of the animal are different.

In an overall and general sense, the invention provides a composition that includes a population of liposomes in which at least one active ingredient is contained substantially within at least a portion of the liposomes, wherein the composition includes from about 20 mole % to about 95 mole % of a first polymerizable lipid, and from about 5 mole % to about 80 mole % of a first unpolymerizable lipid, and wherein at least a first portion of the active ingredient is released from the liposome at a temperature of about 39° C. to about 55° C.

In one embodiment, the invention provides a pharmaceutical composition that comprises, consists essentially of, or alternatively consists of, at least a first population of thermosensitive liposomes and at least a first active ingredient that is contained substantially within the population of liposomes, wherein the composition comprises, consists essentially of, or alternatively consists of, from about 20 mole % to about 90 mole % of a first polymerizable lipid, and from about 80 mole % to about 10 mole % of a first unpolymerizable lipid.

In another embodiment, the composition preferably comprises, consists essentially of, or alternatively consists of, from about 30 mole % to about 80 mole % of a first polymerizable lipid, and from about 70 mole % to about 20 mole % of a first unpolymerizable lipid.

Likewise, the invention also provides thermosensitive liposomal compositions that preferably comprise, consist essentially of, or alternatively consist of, from about 40 mole % to about 70 mole % of a first polymerizable lipid, and from about 60 mole % to about 30 mole % of a first unpolymerizable lipid.

In other embodiments, the invention also provides thermosensitive liposomal compositions that preferably comprise, consist essentially of, or alternatively consist of, from about 50 mole % to about 60 mole % of a first polymerizable lipid, and from about 50 mole % to about 40 mole % of a first unpolymerizable lipid.

In related embodiments, the composition preferably comprises, consists essentially of, or alternatively consists of, (a) from about 35 mole % to about 85 mole % of a first polymerizable lipid, and from about 65 mole % to about 15 mole % of a first unpolymerizable lipid; (b) from about 45 mole % to about 75 mole % of a first polymerizable lipid, and from about 55 mole % to about 25 mole % of a first unpolymerizable lipid, or (c) from about 55 mole % to about 65 mole % of a first polymerizable lipid, and from about 45 mole % to about 35 mole % of a first unpolymerizable lipid.

In exemplary embodiments, the first polymerizable lipid is selected from the group consisting of 23:2 1,2-bis(10,12-tricosadiynoyl)-sn-glycero-3-phosphoethanolamine, 23:2 1,2-bis(10,12-tricosadiynoyl)-sn-glycero-3-phosphocholine, and a combination thereof, while the first unpolymerizable lipid is selected from the group consisting of 22:0 1-stearoyl-2-hydroxy-sn-glycero-3-phosphoethanolamine, 20:0 1-stearoyl-2-hydroxy-sn-glycero-3-phosphoethanolamine, 18:0 1-stearoyl-2-hydroxy-sn-glycero-3-phosphoethanolamine, 16:0 1-stearoyl-2-hydroxy-sn-glycero-3-phosphoethanolamine, 14:0 1-stearoyl-2-hydroxy-sn-glycero-3-phosphoethanolamine, 12:0 1-stearoyl-2-hydroxy-sn-glycero-3-phosphoethanolamine, 10:0 1-stearoyl-2-hydroxy-sn-glycero-3-phosphoethanolamine, 22:0 1,2-bis(10,12-tricosadiynoyl)-sn-glycero-3-phosphocholine, 20:0 1,2-bis(10,12-tricosadiynoyl)-sn-glycero-3-phosphocholine, 18:0 1,2-bis(10,12-tricosadiynoyl)-sn-glycero-3-phosphocholine, 16:0 1,2-bis(10,12-tricosadiynoyl)-sn-glycero-3-phosphocholine, 14:0 1,2-bis(10,12-tricosadiynoyl)-sn-glycero-3-phosphocholine. 12:0 1,2-bis(10,12-tricosadiynoyl)-sn-glycero-3-phosphocholine, 10:0 1,2-bis(10,12-tricosadiynoyl)-sn-glycero-3-phosphocholine, and combinations of two or more thereof.

In one exemplary embodiment, the invention provides a pharmaceutical composition that comprises, consists essentially of, or alternatively consists of: (a) about 40 mole % to about 70 mole % of a first polymerizable lipid selected from the group consisting of 23:2 1,2-bis(10,12-tricosadiynoyl)-sn-glycero-3-phosphoethanolamine and 23:2 1,2-bis(10,12-tricosadiynoyl)-sn-glycero-3-phosphocholine; and (b) about 60 mole % to about 30 mole % of a first unpolymerizable lipid selected from the group consisting of 22:0 1-stearoyl-2-hydroxy-sn-glycero-3-phosphoethanolamine, 20:0 1-stearoyl-2-hydroxy-sn-glycero-3-phosphoethanolamine, 18:0 1-stearoyl-2-hydroxy-sn-glycero-3-phosphoethanolamine, 16:0 1-stearoyl-2-hydroxy-sn-glycero-3-phosphoethanolamine, 14:0 1-stearoyl-2-hydroxy-sn-glycero-3-phosphoethanolamine, 12:0 1-stearoyl-2-hydroxy-sn-glycero-3-phosphoethanolamine, 10:0 1-stearoyl-2-hydroxy-sn-glycero-3-phosphoethanolamine, 22:0 1,2-bis(10,12-tricosadiynoyl)-sn-glycero-3-phosphocholine, 20:0 1,2-bis(10,12-tricosadiynoyl)-sn-glycero-3-phosphocholine, 18:0 1,2-bis(10,12-tricosadiynoyl)-sn-glycero-3-phosphocholine, 16:0 1,2-bis(10,12-tricosadiynoyl)-sn-glycero-3-phosphocholine, 14:0 1,2-bis(10,12-tricosadiynoyl)-sn-glycero-3-phosphocholine. 12:0 1,2-bis(10,12-tricosadiynoyl)-sn-glycero-3-phosphocholine, and 10:0 1,2-bis(10,12-tricosadiynoyl)-sn-glycero-3-phosphocholine, wherein greater than about 50% of the active ingredient is released from the population of liposomes when the liposomes are maintained at a temperature of from about 42° C. to about 45° C. for a period of time of from about 5 to about 30 minutes.

Preferably, in the compositions of the present invention, greater than about 20%, greater than about 30%, greater than about 40%, greater than about 50%, greater than about 60%, greater than about 70%, greater than about 80%, or greater than about 90% or more of the active ingredient(s) present in the composition is released from the population of liposomes when the composition is maintained for a period of time at a temperature that is greater than ambient body temperature of the animal into which the composition is formulated to be administered.

In particular embodiments, preferably greater than about 25%, greater than about 35%, greater than about 45%, greater than about 55%, greater than about 65%, greater than about 75%, greater than about 85%, or greater than about 95% or more of the active ingredient(s) present in the composition is released from the population of liposomes when the composition is maintained for a period of time at a temperature that is greater than ambient body temperature of the animal into which the composition is formulated to be administered.

Preferably the active ingredient(s) present in the composition are released from the population of liposomes when the composition is maintained for a period of time of at least about 2 minutes, at least about 3 minutes, at least about 4 minutes, at least about 5 minutes, at least about 6 minutes, at least about 7 minutes, at least about 8 minutes, at least about 9 minutes, or at least about 10 minutes or longer at a temperature that is greater than ambient body temperature of the animal into which the composition is formulated to be administered.

In particular embodiments, the active ingredient(s) present in the composition are substantially released from the population of liposomes after the composition has been maintained for a period of time of at least about 10 minutes, at least about 12 minutes, at least about 14 minutes, at least about 16 minutes, at least about 18 minutes, or at least about 20 minutes or longer at a temperature that is greater than ambient body temperature of the animal into which the composition is formulated to be administered.

Preferably, the pharmaceutical compositions of the present invention will be employed in an animal, and particularly a mammalian host, in which the liposomes will preferably release at least a first portion of the active ingredient(s) associated with the liposomes, following a period of time at a temperature of about 39° C., about 40° C., about 41° C., about 42° C., about 43° C., about 44° C., or about 45° C. or higher.

In certain embodiments, the pharmaceutical compositions of the present invention will be administered to a selected mammalian, and particularly a human host, in an amount and for a time sufficient to release at least a first portion of the active ingredient(s) associated with the liposomes in at least a first population of cells or a selected tissue or organ contained within the body of the mammal, following a period of time at a temperature of about 40° C., about 45° C., about 50° C., about 55° C., about 60° C., about 65° C., or even about 70° C. or higher.

Preferably, the conditions of mild- to moderate localized hyperthermia employed to facilitate release of the contents of the thermosensitive liposomes into the selected cells or tissues undergoing the hyperthermic conditions will include maintaining the cells or tissues at a temperature of from about 39° C. to about 60° C., of from about 40° C. to about 55° C., of from about 41° C. to about 50° C., or alternatively, at a temperature of from about 42° C. to about 45° C. for a period of time sufficient to release at least a first portion of the active ingredient(s) present in the liposomes contained within the pharmaceutical composition.

In exemplary embodiments, the pharmaceutical compositions of the invention will comprise a first polymerizable lipid component that comprises (a) a hydrophilic head group selected from the group consisting of diethylenetriamine pentaacetic acid, ethylenedinitrile tetraacetic acid, tetraazacylododecane 1,4,7,10-tetraacetic acid and cyclohexane-1,2,-diamino-N,N-diacetatecholesterol; or (b) one or more hydrophobic tail groups comprising a polymerizable functional group selected from the group consisting of diacetylene, olefin, acetylene nitrile, styrene, ester, thiol, amide, α-unsaturated ketone, β-unsaturated ketone, α-unsaturated aldehyde, β-unsaturated aldehyde, or a combination thereof.

In various embodiments, the pharmaceutical compositions of the invention will comprise a first polymerizable lipid component that comprises (a) a hydrophilic head group selected from the group consisting of diethylenetriamine pentaacetic acid, ethylenedinitrile tetraacetic acid, tetraazacylododecane 1,4,7,10-tetraacetic acid and cyclohexane-1,2,-diamino-N,N-diacetatecholesterol; and (b) a hydrophobic tail group comprising a polymerizable functional group selected from the group consisting of diacetylene, olefin, acetylene nitrile, styrene, ester, thiol, amide, α-unsaturated ketone, β-unsaturated ketone, α-unsaturated aldehyde, β-unsaturated aldehyde, or a combination thereof.

In exemplary pharmaceutical compositions, the liposomes of the invention may optionally comprise one or more cholesterols, phosphatidylcholines, phosphatidylglycerols, phosphotidylethanolamines, dioleoyphosphatidylethanolamines, distearoylphosphatidylcholines, dioleoyphosphatidylglycerols, or any combination of two or more thereof.

Likewise, the liposomes of the invention may further optionally comprise one or more lipids or lipid derivatives, including without limitation, a neutral lipid selected from the group consisting of a cephalin, a ceramide, a cerebroside, cholesterol, diacylglycerol, diacylphosphatidylcholine, diacylphosphatidylethanolamine, phosphatidylcholine, phosphatidylethanolamine, a sphingolipid, a sphingomyelin, a tetraether lipid, and any combination of two or more thereof.

In an illustrative embodiment, the pharmaceutical compositions of the invention comprise, consist essentially of, or alternatively consist of: a) a first polymerizable lipid that comprises 20 to 80 mol %, preferably 30 to 70 mol %, more preferably 40 to 60 mol % of a first lipid component that comprises, consists essentially of, or alternatively consists of 23:2 1,2-bis(10,12-tricosadiynoyl)-sn-glycero-3-phosphoethanolamine, 23:2 1,2-bis(10,12-tricosadiynoyl)-sn-glycero-3-phosphocholine, or a combination thereof; and (b) a first unpolymerizable lipid that comprises 80 to 20 mol %, preferably 70 to 30 mol %, more preferably 60 to 40 mol % of a second lipid component that comprises, consists essentially of, or alternatively consists of a compound selected from the group consisting of 22:0 1-stearoyl-2-hydroxy-sn-glycero-3-phosphoethanolamine, 20:0 1-stearoyl-2-hydroxy-sn-glycero-3-phosphoethanolamine, 18:0 1-stearoyl-2-hydroxy-sn-glycero-3-phosphoethanolamine, 16:0 1-stearoyl-2-hydroxy-sn-glycero-3-phosphoethanolamine, 14:0 1-stearoyl-2-hydroxy-sn-glycero-3-phosphoethanolamine, 12:0 1-stearoyl-2-hydroxy-sn-glycero-3-phosphoethanolamine, 10:0 1-stearoyl-2-hydroxy-sn-glycero-3-phosphoethanolamine, 22:0 1,2-bis(10,12-tricosadiynoyl)-sn-glycero-3-phosphocholine, 20:0 1,2-bis(10,12-tricosadiynoyl)-sn-glycero-3-phosphocholine, 18:0 1,2-bis(10,12-tricosadiynoyl)-sn-glycero-3-phosphocholine, 16:0 1,2-bis(10,12-tricosadiynoyl)-sn-glycero-3-phosphocholine, 14:0 1,2-bis(10,12-tricosadiynoyl)-sn-glycero-3-phosphocholine. 12:0 1,2-bis(10,12-tricosadiynoyl)-sn-glycero-3-phosphocholine, 10:0 1,2-bis(10,12-tricosadiynoyl)-sn-glycero-3-phosphocholine, and any combination of two or more thereof.

In exemplary embodiments, the first polymerizable lipid comprises, consists essentially of, or alternatively consists of, approximately 20 to 40 mol % of 23:2 1,2-bis(10,12-tricosadiynoyl)-sn-glycero-3-phosphoethanolamine or 23:2 1,2-bis(10,12-tricosadiynoyl)-sn-glycero-3-phosphocholine; and the first unpolymerizable lipid comprises approximately 80 to 60 mol % of 18:0 1-stearoyl-2-hydroxy-sn-glycero-3-phosphoethanolamine or 14:0 1,2-bis(10,12-tricosadiynoyl)-sn-glycero-3-phosphocholine. In another exemplary embodiment, the first polymerizable lipid includes approximately 85 mol % of 23:2 1,2-bis(10,12-tricosadiynoyl)-sn-glycero-3-phosphoethanolamine (23:2 diyne-PE) or 1,2-bis(10,12-tricosadiynoyl)-sn-glycero-3-phosphocholine (23:2 diyne-PC), and the first unpolymerizable lipid includes approximately 15 mol % of 18:0 1-stearoyl-2-hydroxy-sn-glycero-3-phosphoethanolamine (18:0 lyso-PE) or 14:0 1,2-bis(10,12-tricosadiynoyl)-sn-glycero-3-phosphocholine (14:0 lyso-PC), or any combination thereof.

In the practice of the invention, it is desirable that the thermosensitive liposomes are adapted and configured to release at least a first portion of the one or more active ingredient(s) contained therein, by application of a suitable energy source to induce at least a first localized region of hyperthermia. Such energy sources include, without limitation, the external application of heat, ultrasound energy, high intensity focused ultrasound energy, laser energy, photoacoustic energy, ultrasonographic energy, radiant energy, light energy, or placement of the region of interest in a magnetic field, and such like.

In certain embodiments, it may be desirable to associate the active ingredient(s) within or about the lipid bilayer membranes that comprise the liposomes, or alternatively, to encase, entrap, or encapsulate the active ingredient(s) substantially within the interiors (i.e., the lumen) of the liposomes themselves. Preferably, at least about 40 percent, at least about 50 percent, at least about 60%, at least about 70%, at least about 80%, at least about 90% or more of the active ingredient(s) is contained substantially within the lumen of the liposome, or associated within or about the lipid bilayer membranes that comprise the liposomes. In some applications, it may be desirable to associate a first active ingredient within or about the lipid bilayer membranes that comprise the liposomes, which a second active ingredient (or plurality of active ingredients) is contained substantially within the lumen of the liposomes. An exemplary application includes, for example, association of one or more detectable labels within or about the lipid bilayer, to facilitate detection of the composition, and encapsulation of one or more pharmaceutical agents within the lumen of the liposomes.

As noted herein, the thermosensitive liposomes of the invention will preferably comprise one or more diagnostic or therapeutic agents, including, without limitation, one or more active ingredient(s) selected from the group consisting of an antineoplastic agent, an immunomodulating agent, a neuroactive agent, an antiflammatory agent, an antilipidemic agent, a hormone, a receptor agonist or antagonist, an antiinfective agent, a protein, a peptide an antibody, an enzyme, an RNA, a DNA, an siRNA, an mRNA, a ribozyme, a hormone, a cofactor, a steroid, an antisense molecule, a detection agent, an imaging agent, a contrast agent, a gas, a pharmaceutically-active molecule, and any combination of two or more thereof.

Preferably the thermosensitive liposomes of the present invention are stable at a pH of from about 4.0 to about 8.0, preferably at a pH of from about 4.5 to 7.5, and more preferably at a pH of from about 5 to about 7.

The liposomes of the present invention may further optionally comprise one or more detectable labels, including, without limitation, fluorogenic, radioactive, chemiluminescent, and photoluminescent labels, and particularly those having applicability and commercial availability for the medical, diagnostic, and therapeutic arts. Such labels may be associated, integrated with, or bound to the liposomes themselves, or alternatively, may comprise, or be associated with, or chemically coupled to, the active ingredient(s) contained within the liposomes.

In the practice of the invention, it is desirable that the majority of the liposomes in a particular population of thermosensitive liposomes will be of sufficient diameter to afford delivery of the active ingredient(s) to the selected cells or tissues. As such, it is contemplated that in routine formulation of the disclosed compositions, a majority of the liposomes in a particular population of thermosensitive liposomes will have a nominal diameter of about 10 nm to about 10 μm, preferably about 50 nm to about 5 μm, more preferably about 100 nm to about 1 μm, although liposomes of any practical size are contemplated to fall within the scope of the disclosure, including without limitation, all intermediate integers in the recited ranges.

As noted herein, the pharmaceutical compositions of the invention may further optionally comprise one or more pharmaceutically-acceptable buffers, diluents, vehicles, or any combination thereof, and may preferably be formulated for administration to an animal host cell, and to mammalian host cells, such as human host cells in particular. In certain embodiments, the compositions may also further optionally comprise one or more compounds selected from the group consisting of surfactants, niosomes, ethosomes, transferosomes, phospholipids, sphingosomes, and any combination thereof.

The pharmaceutical compositions of the invention will find use in therapy, particularly in hyperthermic therapy, photoablation, photothermal, photoacoustic, ultrasound, high intensity focused ultrasound, laser therapy, and any combination thereof.

Likewise, in certain embodiments, the pharmaceutical compositions of the invention will find use in diagnosis, particularly in the detection, diagnosis, identification, or monitoring of one or more diseases, disorders, dysfunctions, abnormal conditions, or trauma. In particular embodiments, the pharmaceutical compositions of the invention will find particular use in medical imaging, including without limitation, computer-assisted tomographic (CT) imaging, ultrasonography, magnetic resonance imaging (MRI), and the like.

In one embodiment, the invention concerns a method of providing a therapeutic or diagnostic compound to a first cell in an animal. In an overall and general sense, the method generally involves administering to one or more cells, tissues, organs, or to the circulatory system of an animal, a therapeutically-or diagnostically-effective amount of one or more of the thermosensitive liposomal formulations as disclosed herein.

The invention further provides a method for administering a first prophylactic compound to a first cell or tissue site within the body of an animal. In an overall and general sense, the method generally involves providing to the animal a prophylactically-effective amount of one or more active ingredient(s) prepared in a liposomal formulation as disclosed herein.

Likewise, the invention provides a method for administering a first diagnostic agent or imaging reagent to a selected population of cells or to a first selected tissue or organ within the body of a mammal. In an overall and general sense, such a method generally involves providing to the mammal (either systemically, or directly or indirectly to one or more selected cells, tissues, or organs within the mammal) an effective amount of one or more active ingredient(s) contained within one of the thermosensitive liposomal compositions disclosed herein, under conditions and for a period of time effective to release at least a first portion of the diagnostic agent or imaging reagent substantially only in the target cell or tissue, without releasing a substantial amount of the diagnostic agent or imaging reagent in non-selected cells or tissues of the subject to which the composition is administered.

In exemplary embodiments, at least a first portion of the diagnostic agent or imaging reagent may be released substantially only in the target cell or tissue, by the localized application of one or more forms of thermal, ultrasound, or magnetic field-inducing energy to the target cell or tissue in an amount effective to release at least about 30%, at least about 40%, at least about 50%, at least about 60%, at least about 70% or at least about 80% or more of the diagnostic agent or imaging reagent contained within the population of liposomes into the target cells, tissue, or organs of interest.

Release of the active ingredient(s) from the liposomes according to the present invention may also optionally be facilitated by heat, ultrasound, laser, magnetic, photoacoustic, and/or light energy (including visible (VIS), infrared (IR), near-infrared (NIR), ultraviolet (UV), and near-UV (NUV), or a combination thereof, or by any other means that results in the “leaking” (i.e., at least partial release) of the active ingredient(s) from the liposomes.

Exemplary active ingredient encapsulated by the liposomal delivery agents may include, but are not limited to, one or more antineoplastic agents, immunomodulating agents, neuroactive agents, antiflammatory agents, chemotherapeutic agents, antilipidemic agents, hormones, trophic factors, cytokines, receptor agonists or antagonists, antimicrobial agents (including antibacterials, antifungals, antiamebics, antihelminthics, and antivirals), antiinfective agents, or such like, or any combination thereof.

Exemplary active ingredients include, but are not limited to, one or more ingredients including a protein, a peptide, or polypeptide (including, for example, without limitation, enzymes, antibodies, antigens, antigen binding fragments etc.); an RNA molecule (including siRNA, mRNA, tRNA, antisense oligonucleotides, and polynucleotides, as well as catalytic RNAs such as ribozymes, and the like); a DNA molecule (including for example, without limitation, oligonucleotides, polynucleotides, genes, plasmids, vectors, and such like); peptide nucleic acids, a viral particle, vector, or virion; a detection agent, an imaging agent, a contrast agent, a detectable gas, or such like, and a pharmaceutically-active molecule, including one or more drugs, prodrugs, cofactors, hormones, steroids, etc., or any combination thereof.

Preferably, the liposomes will have an isoelectric point from about 5.5 to about 8.5, more preferably from about 6 to about 8, and still more preferably from about 6.5 to about 7.5. In most applications of the present methods, the compositions as disclosed herein will be at least substantially stable at a pH from about 4.2 to about 8.2, and more preferably, will be substantially stable at a pH of from about 5 to about 7.5. Preferably, the active ingredient(s) and delivered agent(s) contained within the thermoactivatable liposomal preparations described herein will be substantially active at physiological conditions of the animal into which they are being administered.

Preferably, the liposome compositions of the present invention will be formulated such that at least about 60, about 65, about 70 or about 75 percent or more of the active ingredient(s) will be initially contained substantially within the interior of the liposomes before the leaking of the active ingredient(s) is initiated. More preferably, the liposome compositions of the present invention will be formulated such that at least about 80, about 85, about 90, or even about 95 percent or more of the active ingredient(s) will be contained substantially within the interior of the liposomes before the leaking is initiated.

The compositions of the present invention may also further optionally include one or more neutral lipids, including those selected from the group consisting of cephalin, ceramide, cerebrosides, cholesterol, diacylglycerol, diacylphosphatidylglycerol diacylphosphatidylcholine, diacylphosphatidylethanolamine, phosphatidylethanolamines, phosphatidylcholine, phosphatidylethanolamines, sphingolipid, sphingomyelins, tetraether lipids, or any combination thereof.

The liposomes of the present invention will preferably have an average diameter of about 10 nm to about 10 μm, more preferably from about 50 nm to about 1 μm, and more preferably still, from about 100 nm to about 800 nm. In certain embodiments, the liposomes will have an average diameter of about 150, about 200, about 250, about 300, about 350, about 400 nm, about 450 nm, or even about 500 nm or more while in other embodiments, the liposomes may be substantially larger than those dimensions, even up to an including liposomes having an average diameter of about 1 μm, about 2 μm, about 3 μm, about 4 μm, or even about 5 μm or more. Alternatively, in some applications of the invention, substantially smaller average diameter liposomes may be formulated, including, for example, without limitation, those with an average diameter of about 20 nm, about 30 nm, about 40 nm, about 50 nm, about 60 nm, about 70 nm, about 80 nm, about 90 nm, or even about 10 nm or more. In certain applications, at least substantially all of the liposomes in a given formulation will be of similar average diameters, while in other applications it may be advantageous or desirable to prepare formulations of liposomes having at least two distinct average diameters, and in some instances, even pluralities of liposomes with substantially different average diameters.

The compositions of the present invention may further optionally include one or more surfactants, lipid complexes, niosomes, ethosomes, transferosomes, phospholipids, sphingolipids or sphingosomes, and may optionally be encompassed within a nanoparticle, a microparticle, a nanocapsule, a microcapsule, a nanosphere, a microsphere, or a combination thereof. Preferably, the disclosed liposomal compositions will generally be formulated for administration to an animal host cell, and in particular, to a mammalian host cell such as a human.

The present invention provides compositions for use in therapy, prophylaxis, and/or diagnosis including, but not limited to, photoablation, laser, thermal, acoustic, photoacoustic, magnetic, and/or ultrasound, therapy, or any combination thereof. The present invention also provides compositions for use in diagnosis, including, for example, without limitation, the diagnosis of disease via one or more diagnostic imaging modalities (including, for example, without limitation, computer-assisted tomographic [CT] imaging, ultrasonography, magnetic resonance imaging [MRI], and the like). The composition may include one or more detectable labels or gases, diagnostic markers, imaging or contrast agents, radiolabeled compound, fluorogenic substance, chemiluminescent or bioluminescent molecule, or any other suitable active ingredient(s) or combinations thereof that may be employed in one or more diagnostic methodologies available in the art.

The present invention also provides for the use of one or more of the disclosed liposomal compositions in the manufacture of a medicament for diagnosis, prophylaxis or therapy, and particularly for use in the manufacture of a medicament for diagnosis, treating, and/or preventing one or more diseases, dysfunctions, conditions, or disorders, or the symptoms thereof, in a mammal, and in a human in particular.

The present invention also provides for the use of one or more of the disclosed liposomal compositions in the manufacture of a medicament for diagnosis, prophylaxis or therapy of one or more medical conditions, including for example, cancer; diabetes; neurological disorders; cerebrovascular accidents; stroke, ischemia, infarction, aneurysm, musculoskeletal deficiencies; neuromuscular disorders; peptide, polypeptide, or enzyme deficiencies; hormone, cofactor, or trophic factor deficiency; cardiovascular and/or cardiocirculatory disease, disorder, or dysfunction; organ disease, dysfunction, or failure; genetic disorders; congenital abnormalities, defects, or malformations; trauma; or symptoms thereof.

The present invention also provides for the use of one or more of the disclosed liposomal compositions in the manufacture of a medicament for the prevention of disease, including, in the preparation of one or more vaccines suitable for prophylactic administration.

The invention also provides methods for providing a therapeutic, prophylactic, or diagnostic compound to a first cell in a mammal, with the method generally including providing to a mammal in need thereof, an effective amount of a liposomal composition as disclosed herein that includes at least one therapeutic, prophylactic, or diagnostic active ingredient, and for a time effective to provide the desired therapy, prophylaxis or diagnosis in the selected mammal.

In certain aspects of the invention, the invention provides liposomal compositions for delivering one or more compounds to a host cell. In particular embodiments, the host cell is a mammalian host cell. In certain preferred embodiments of the invention, the host cell is a human cell. In other preferred aspects, the host cell is included within the body of a human, or included within at least a first ex vivo tissue or plurality of cells that are compatible for implantation into the body of such a human as part of a typical ex vivo therapy protocol or such like.

In other aspects of the invention, liposomal compositions are provided that may further optionally include a third distinct lipid component, and/or a second distinct agent encapsulated in the liposome for delivery. In some aspects of the invention, it may be desirable to prepare liposome-within-a-liposome formulations in which a first active ingredient is entrapped within a first liposomal formulation, and the first liposomal formulation is then entrapped within a second distinct liposome, either alone, or in concert with a second distinct active ingredient. Such combination approaches may be particularly desirable when it is desirable to provide a single injection into the animal, in which one active ingredient is released from the “outer” liposome at a particular temperature, and then the second active ingredient is released from the “inner” liposome at a distinctly higher temperature. In these instances, the active ingredients can be two distinct imaging agents, two distinct therapeutic agents, two distinct prophylactic agents, or a combination of one or more of such agents.

Similarly, the disclosed thermally-activatable liposomal compositions may also be formulated to include one or more additional vehicles for delivering an agent to an animal, including, for example, without limitation, fully-polymerized liposomes, partially-polymerized liposomes, cross-linked lipids, unpolymerized liposomes, niosomes, transferosomes, ethosomes, phospholipid complexes, lipid particles, lipid vesicles, nanoparticles, microparticles, nanocapsules, microcapsules, microspheres, nanospheres, sphingosomes, and such like.

A variety of methods are available for preparing liposomes and well known to those of ordinary skill in the art (see e.g., U.S. Pat. Nos. 7,273,620, 5,720,976, 6,200,598, 6,726,925, and 6,964,778, each of which is specifically incorporated herein in its entirety by express reference thereto). The liposomal compositions of the invention could also be delivered in association with a conventional pharmaceutical compound or composition, which preferably may provide diagnosis, prophylaxis, and/or treatment of the same (or different) type as provided by the liposomal composition.

### Therapeutic, Prophylactic and Diagnostic Methods

Another important aspect of the present invention concerns methods for using the disclosed liposome compositions to deliver one or more therapeutic agents for treating or ameliorating the symptoms of disease, disorder, dysfunction, abnormal condition, trauma, deficiency, or symptoms thereof in a mammal. Such methods generally involve administration to a mammal, or human in need thereof, one or more of the disclosed compositions, in an amount and for a time sufficient to treat or ameliorate one or more symptoms of such a disease, deficiency, disorder, dysfunction, or abnormal condition in the affected mammal. The methods may also encompass prophylactic treatment of animals suspected of having such diseases, disorders, conditions, dysfunctions, or deficiencies, and administration of such compositions to those animals diagnosed with a symptom of, or at risk for developing, one or more such diseases, disorders, conditions, dysfunctions, or deficiencies either following diagnosis, or prior to the onset of at least one symptom of such a condition.

As such, preferred animals for administration of the pharmaceutical compositions disclosed herein include mammals, and particularly humans. Other preferred animals include non-human primates, bovines, ovines, caprines, lupines, equines, porcines, canines, and felines, as well as animals under the care of a veterinary practitioner.

### Pharmaceutical Formulations

In certain embodiments, the present invention concerns formulation of one or more therapeutic or diagnostic agents in a pharmaceutically acceptable liposome composition for administration to a cell or an animal, either alone, or in combination with one or more other modalities of prophylaxis and/or therapy. The formulation of pharmaceutically-acceptable excipients and carrier solutions is well known to those of ordinary skill in the art, as is the development of suitable dosing and treatment regimens for using the particular compositions described herein in a variety of treatment regimens.

In certain circumstances it will be desirable to deliver the thermally-activatable partially-polymerized liposome-based compositions in suitably-formulated pharmaceutical vehicles by one or more standard delivery means, including, for example, without limitation, subcutaneously, intraocularly, intravitreally, parenterally, intravenously, intracerebroventricularly, intramuscularly, intrathecally, orally, intraperitoneally, transdermally, topically, by oral or nasal inhalation, or by direct injection to one or more cells, tissues, or organs. The methods of administration may also include those modalities as described in U.S. Pat. Nos. 5,543,158; 5,641,515, and 5,399,363, each of which is specifically incorporated herein in its entirety by express reference thereto). Solutions of the active compounds as freebase or pharmacologically acceptable salts may be prepared in sterile water, and may be suitably mixed with one or more surfactants, such as hydroxypropylcellulose. Dispersions may also be prepared in glycerol, liquid polyethylene glycols, oils, or mixtures thereof. Under ordinary conditions of storage and use, these preparations contain a preservative component to prevent or retard the growth of contaminants, including, without limitation, microorganisms and the like.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, a sterile aqueous medium that can be employed will be known to those of ordinary skill in the art in light of the present disclosure. For example, one dosage may be dissolved in 1 mL of isotonic NaCl solution, and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will determine, in any event, the appropriate dose for the individual subject. Moreover, for human administration, preparations should meet sterility, pyrogenicity, and the general safety and purity standards as required by FDA Office of Biologics standards.

Sterile injectable compositions may be prepared by incorporating the disclosed liposomal-vectored diagnostic or therapeutic compounds in the required amount in the appropriate solvent with several of the other ingredients enumerated above, as required, followed by filtered sterilization. Generally, dispersions can be prepared by incorporating the selected sterilized active ingredient(s) into a sterile vehicle that contains the basic dispersion medium and the required other ingredients from those enumerated above. The compositions disclosed herein may also be formulated in a neutral or salt form. Pharmaceutically-acceptable salts include the acid addition salts (formed with the free amino groups of the protein), and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation, and in such amount as is effective for the intended application. The formulations are readily administered in a variety of dosage forms such as injectable solutions, topical preparations, oral formulations, including sustain-release capsules, hydrogels, colloids, viscous gels, transdermal reagents, intranasal and inhalation formulations, and the like.

The amount of liposome-mediated composition(s) and the time needed for the administration of such composition(s) will be within the purview of the ordinary-skilled artisan having benefit of the present teachings. It is likely, however, that the administration of a therapeutically-effective, pharmaceutically-effective, prophylactically-effective, and/or diagnostically-effective amount of the disclosed liposome compositions may be achieved by a single administration, such as for example, a single injection of a sufficient quantity of the delivered agent to provide the desired benefit to the patient undergoing such a procedure. Alternatively, in some circumstances, it may be desirable to provide multiple, or successive administrations of the liposome-vectored pharmaceutical or diagnostic compositions, either over a relatively short, or even a relatively prolonged period of time, as may be determined by the medical practitioner overseeing the administration of such compositions to the selected individual.

Typically, formulations of one or more active ingredients in the liposomal formulations disclosed herein will contain an effective amount for the selected therapy or diagnosis. Preferably, the formulation may contain at least about 0.1% of each active ingredient, although the percentage of the active ingredient(s) may, of course, be varied, and may conveniently be present in amounts from about 0.5 to about 80 weight % or volume %, or from about 1 to about 70 weight % or volume %, or more preferably, from about 2 to about 50 weight % or volume %, based upon the total formulation. Naturally, the amount of active compound(s) in each liposomal composition may be prepared in such a way that a suitable dosage will be obtained in any given unit dose of the compound. Factors such as solubility, bioavailability, biological t_{1/2}, route of administration, product shelf life, as well as other pharmacological considerations will be contemplated by one of ordinary skill in the art of preparing such pharmaceutical formulations, and as such, a variety of dosages and treatment regimens may be desirable.

The pharmaceutical compositions disclosed herein may be administered parenterally, intravenously, intramuscularly, or even intraperitoneally as described in U.S. Pat. Nos. 5,543,158, 5,641,515 and 5,399,363, each of which is specifically incorporated herein in its entirety by express reference thereto). Solutions of the active compounds as free-base or pharmacologically acceptable salts may be prepared in water suitably mixed with a surfactant, such as hydroxypropylcellulose, or other similar fashion. The pharmaceutical forms adapted for injectable administration include sterile aqueous solutions or dispersions, and sterile powders for the extemporaneous preparation of sterile injectable solutions or dispersions including without limitation those described in U.S. Pat. No. 5,466,468, which is specifically incorporated herein in its entirety by express reference thereto). In all cases, the form must be sterile and must be fluid to the extent that easy syringability exists. It must be at least sufficiently stable under the conditions of manufacture and storage, and must be preserved against the contaminating action of microorganisms, such as viruses, bacteria, fungi, and such like. The carrier can be a solvent or dispersion medium containing, for example, water, ethanol, polyol (e.g., glycerol, propylene glycol, and liquid polyethylene glycol, and the like, or a combination thereof), one or more vegetable oils, or any combination thereof, although additional pharmaceutically-acceptable components may be included.

Proper fluidity may be maintained, for example, by the use of a coating, such as e.g., a lecithin, by the maintenance of the required particle size in the case of dispersion, by the use of a surfactant, or any combination of these techniques. The inhibition or prevention of the action of microorganisms can be brought about by one or more antibacterial or antifungal agents, for example, without limitation, a paraben, chlorobutanol, phenol, sorbic acid, thimerosal, or the like. In many cases, it will be preferable to include an isotonic agent, for example, one or more sugars or sodium chloride, or any combination thereof. Prolonged absorption of the injectable compositions can be brought about by the use in the compositions of agents delaying absorption, for example without limitation, aluminum monostearate, gelatin, or a combination thereof.

Sterile injectable solutions of the disclosed liposomal-vectored compounds may be prepared by incorporating the active compound(s) in the required amount into the appropriate amount of the liposomes, either alone or in combination with one or more other components. Generally, dispersions are prepared by incorporating the various sterilized active ingredients into a sterile vehicle which contains the basic dispersion medium and the required other ingredients from those enumerated above. In the case of sterile powders for the preparation of sterile injectable liposomal solutions, the preferred methods of preparation are vacuum drying and freeze-drying techniques, which yield a powder of the active ingredient plus any additional desired ingredient from a previously sterile-filtered solution thereof.

It is contemplated that formulations of the disclosed liposome-based agent delivery compositions may be suitable for direct injection into one or more organs, tissues, or cell types in the body. Such injection sites include, but are not limited to, the circulatory system, the spinal cord, the lymphatic system, a joint or joint capsule, a synovium or subsynovium tissue, tendons, ligaments, cartilages, bone, periarticular muscle or an articular space of a mammalian joint, as well as direct administration to an organ or tissue site such as the heart, liver, lung, pancreas, intestine, brain, bladder, kidney, or other site within the patient's body, including, for example, without limitation, introduction of the delivered therapeutic or diagnostic agent(s) via intra-abdominal, intra-thoracic, intravascular, or intracerebroventricular delivery of a suitable liposomal formulation. Administration of the disclosed compositions need not be restricted to one or more of these delivery means, but instead may be conducted using suitable means, including those known to the one of ordinary skill in the relevant medical arts. In certain embodiments the active ingredients of the invention may be formulated for delivery by needle, catheter, and related means, or alternatively, may be included within a medical device, including, for example, without limitation, drug-eluting implants, stents, catheters, and such like. The formulations may also be prepared for injection by an implanted drug-delivery pump or similar mechanism.

The administration of the pharmaceutical compositions by intranasal sprays, inhalation, and/or other aerosol delivery vehicles is also contemplated. Methods for delivering genes, nucleic acids, and peptide compositions directly to the lungs via nasal aerosol sprays has been described e.g., in U.S. Pat. Nos. 5,756,353 and 5,804,212, each of which is specifically incorporated herein in its entirety by express reference thereto. Delivery of drugs using intranasal microparticle resins (see e.g., Takenaga et al., 1998) and lysophosphatidyl-glycerol compounds (U.S. Pat. No. 5,725,871, specifically incorporated herein in its entirety by express reference thereto) are also well-known in the pharmaceutical arts. Likewise, transmucosal drug delivery in the form of a polytetrafluoroethylene support matrix is suitable in the practice of the invention, and is described in U.S. Pat. No. 5,780,045 (specifically incorporated herein in its entirety by express reference thereto).

The disclosed liposome formulations may also be administered through transdermal or other topical administration routes. Exemplary methods for the use of liposomal formulations in topical therapy are found, for example, in U.S. Pat. Nos. 5,540,936, and 6,133,451, each of which is specifically incorporated herein in its entirety by express reference thereto.

In particular embodiments, the disclosed liposomal compositions can be formulated using one or more pharmaceutical buffers, vehicles, or diluents, and intended for administration to a mammal through suitable means, such as, by intramuscular, intravenous, subcutaneous, intrathecal, intra-abdominal, intravascular, intra-articular, or alternatively, by direct injection to one or more cells, tissues, or organs of such a mammal.

The liposomal-based pharmaceutical formulations disclosed herein are not in any way limited to use only in humans, or even to primates, or mammals. In preferred embodiments, however, the compositions of the present invention may be formulated for administration to a mammal, including humans, for a variety of diagnostic, therapeutic, and/or prophylactic regimens. Such liposomes may also be provided in excipient formulations that are acceptable for veterinary administration, including, for example, without limitation, to selected livestock, exotic or domesticated animals, companion animals (including pets and such like), non-human primates, as well as zoological or otherwise captive specimens, and such like.

Such methods may also encompass prophylactic treatment of one or more animals suspected of having, or at risk for developing one or more such conditions either following diagnosis, or prior to the onset of symptoms. To that end, in certain embodiments the liposomal compositions disclosed and/or described herein may also find utility in the area of vaccine development, and antigen administration/vaccination and the like.

### Compositions for the Preparation of Medicaments

Another important aspect of the present invention concerns methods for using the disclosed liposomal compositions (as well as formulations including them) in the preparation of medicaments for preventing, treating or ameliorating the symptoms of various diseases, dysfunctions, or deficiencies in an animal, such as a vertebrate mammal. Use of the disclosed liposomal compositions is also contemplated in therapy and/or prophylaxis of one or more diseases, disorders, dysfunctions, conditions, disabilities, deformities, or deficiencies, particularly when the liposomal vehicle is formulated to include one or more therapeutic (or prophylactic) agents known to one of ordinary skill in the medical arts.

Such use generally involves administration to an animal in need thereof one or more of the disclosed liposomal compositions that include at least a first therapeutic or prophylactic agent, in an amount and for a time sufficient to prevent, treat, lessen, or ameliorate one or more symptoms of such a disease, disorder, dysfunction, condition, disability, deformity, or deficiency in the affected animal.

Compositions including one or more of the disclosed liposomal formulations also form part of the present invention, and particularly those compositions that further include at least a first pharmaceutically-acceptable excipient for use in the therapy, prophylaxis, or diagnosis of one or more diseases, dysfunctions, disorders, or such like.

Use of the disclosed compositions is also contemplated, particularly in the manufacture of medicaments and methods involving one or more therapeutic (including chemotherapy, phototherapy, laser therapy, etc.) prophylactic (including e.g., vaccines), or diagnostic regimens, (including, for example, without limitation, in diagnostic imaging methods, including, without limitation, ultrasound, HIFU, CT, MRI, and the like).

Such liposomal formulations may optionally further include one or more additional distinct lipids, lipid complexes, phospholipids, liposomes, or other such vehicles, additives or adjuvants as may be suitable for administration to an animal. Such routes of administration are known to and may be selected by those of ordinary skill in the art, and include, for example, but are not limited to, delivery means including intramuscular, intravenous, intra-arterial, intrathecal, intracavitary, intraventricular, subcutaneous, or direct injection into an organ, tissue site, or population of cells in the recipient animal.

### BRIEF DESCRIPTION OF THE DRAWINGS

For promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

The following drawings form part of the present specification and are included to demonstrate certain aspects of the present invention. The invention may be better understood by reference to the following description taken in conjunction with the accompanying drawings, in which like reference numerals identify like elements, and in which:

FIG. 1 illustrates exemplary lipid compounds for use in formulating the liposomes of the present invention. Exemplary polymerizable lipids include 23:2 Diyne-PE (Compound “A”), and 23:2 Diyne-PC (Compound “D”), while exemplary non-polymerizable lipids include 18:0 Lyso-PE (Compound “B”), 14:0 Lyso-PE (Compound “C”), 25:0 Lyso-PE (Compound “E”), 10:0 Lyso-PE (Compound “F”), and 6:0 Lyso-PE (Compound “G”);

FIG. 2 illustrates examples of small-molecular weight molecules: Texas Red sulfonyl chloride mixed isomers (Sigma Chemical Corp., St. Louis, Mo., USA; hereinafter “Texas Red”), and 5-carboxyfluorescein (hereinafter “5-CF”) that may be encapsulated in the thermosensitive liposome formulations described herein to illustrate the thermoregulatible release of molecules contained within the liposomes;

FIG. 3 illustrates the overall experimental design for formation of thermosensitive liposomal complexes in accordance with one aspect of the invention. Shown here are polymerizable lipids (compounds A and B) reacted under UV conditions with a non-polymerizable lipid (compound B) to produce a thermally-controlled release liposomal composition;

FIG. 4A and FIG. 4B illustrate exemplary results obtained in the formation of thermosensitive liposomal complexes in accordance with one aspect of the invention. By varying the content of the non-polymerizable lipid in the liposomal complex, the quantity of molecules in the lumen of the liposomes released at a given temperature may be controlled. As observed in both FIG. 4A and FIG. 4B, at normal human body temperature (37° C.), virtually none of the molecules contained with the liposomes (here a fluorescent dye) is released over time, resulting in a very stable liposome, with little release of its contents over time. However, when the temperature is raised (e.g., 42° C. or 45° C.), the liposomes become “leaky” and their contents are then released over time. Comparing FIG. 4A (in which the hybrid liposomes were formulated using a relatively low amount of non-polymerizable lipid) and FIG. 4B (in which the hybrid liposomes were formulated using a relatively high amount of non-polymerizable lipid), the effect of changing the ratio of the lipid components in the liposomal formulation can clearly be seen;

FIG. 5A and FIG. 5B illustrate exemplary results obtained in the formation of thermosensitive liposomal complexes in accordance with one aspect of the invention. By varying the content of the non-polymerizable lipid in the liposomal complex, molecules of particular molecular weight ranges in the lumen of the liposomes may be preferably released at a given temperature. As observed in both FIG. 5A and FIG. 5B, at normal human body temperature (37° C.), substantially neither a small-molecular-weight compound (FIG. 5A, the fluorescent dye Texas Red, MW=625.15) or a large-molecular-weight compound (FIG. 5B, the fluorescent dye Texas Red coupled to Dextran, MW=3000) is released over time. However, when the temperature is raised (e.g., to either 42° C. or 45° C.), the liposomes become “leaky” and their contents are then released over time. Comparing FIG. 5A (in which the hybrid liposomes contain relatively large molecules) and FIG. 5B (in which the hybrid liposomes contain relatively small molecules), the effect of changing the ratio of the lipid components in the liposomal formulation can clearly be seen;

FIG. 6 illustrates results of release of 5-CF molecules from an exemplary thermosensitive liposome formulation in accordance with one aspect of the invention over time at either 37° C. or 45° C. at two different concentrations (10 and 100 μM) of active ingredient;

FIG. 7A, FIG. 7B, FIG. 7C, FIG. 7D, FIG. 7E, and FIG. 7F illustrate results of release of Texas Red molecules (MW=625.15) from exemplary thermosensitive liposome formulations in accordance with one aspect of the invention over time at various temperatures. With approximately 40 mol % of polymerizable lipids included within the liposomes, the 625.15-MW molecule could be effectively released from the liposomes at 42° C. but not at 37° C. When no polymerizable lipids were included within the liposome formulation, even relatively small-molecular-weight molecules (e.g., Texas Red, MW=625.15) were not effectively released at 42° C. or 37° C. The percent non-polymerizable lipid present in each formulation varies from 0% to 28% over FIG. 7A to FIG. 7F as shown;

FIG. 8A, FIG. 8B, and FIG. 8C illustrate results of release of Dextran/Texas Red molecules (MW=3000) (FIG. 8A) and Texas Red molecules (MW=625.15) (FIG. 8B and FIG. 8C) from various liposome formulations over time at 37° C. and 45° C. With approximately 15 mol % of unpolymerizable lipids (Compound B; FIG. 8B, and Compound C; FIG. 8C) included in the liposomes, small MW molecules were significantly released at both 42° C. and 37° C. However, with approximately 15 mol % of unpolymerizable lipids (Compound B) included in the liposome formulation (FIG. 8A), large MW molecules were substantially released at 45° C., but only slightly so at 37° C.;

FIG. 9 summarizes the percent release (as determined by RadioTLC) of the radioactive compound, ^{99m}Tc-HIDA, from thermosensitive liposome formulations incubated for 5 minutes after purification (at either 37° C., 48° C., or 74° C.) that were formulated with varying percentage of unpolymerizable lipid (Compound B). With successively higher content of unpolymerizable lipid, the liposomes became less stable and more of the active ingredient is released;

FIG. 10 illustrates results of release of Dextran/Texas Red molecules (MW=3000) from liposome formulations over time at various temperatures. With approximately 50 mol % of unpolymerizable lipids (Compound B) included in the liposomes large MW molecules can be released at 45° C., but not at 42° C. or 37° C.;

FIG. 11A and FIG. 11B illustrate results of release of Texas Red molecules (MW=625.15) from liposome formulations over time at various temperatures. With approximately 40 mol % of unpolymerizable lipids (Compound B) included in the liposomes (FIG. 11A), small MW molecules were released at 42° C., but not at 37° C. With no unpolymerized lipids present in the liposome formulation, small molecules were not released at either 42° C. or 37° C. (FIG. 11B);

FIG. 12A and FIG. 12B illustrate results of release of Texas Red molecules (MW=625.15) from liposome formulations over time at various temperatures. With approximately 60 mol % of unpolymerizable lipids included in the liposomes (FIG. 12A), the release of small MW molecules was much faster than with 40 mol % unpolymerizable lipids (FIG. 12B);

FIG. 13 illustrates release of Dextran/Texas Red molecules (MW=3000) from thermosensitive liposome formulations over time at various temperatures. With approximately 50 mol % of unpolymerizable lipids (Compound B) included in the liposomes large MW molecules can be released at 45° C., but not at 42° C. or 37° C.;

FIG. 14A and FIG. 14B illustrate release of Texas Red molecules (MW=625.15) from thermosensitive liposome formulations over time at various temperatures. With approximately 40 mol % of unpolymerizable lipids (Compound B) included in the liposomes (FIG. 14A), small MW molecules were released at 42° C., but not at 37° C. With no unpolymerized lipids present in the liposome formulation, small molecules were not released at either 42° C. or 37° C. (FIG. 14B);

FIG. 15A and FIG. 15B illustrate release of Texas Red molecules (MW=625.15) from thermosensitive liposome formulations over time at various temperatures. With approximately 37 mol % of unpolymerizable lipids (Compound B) included in the liposomes (FIG. 15A), small MW molecules were released at 45° C. and 42° C., but not at 37° C. With 60 mol % of unpolymerized lipids (Compound B) present in the liposome formulation, small molecules were released at 42° C. but not 37° C. (FIG. 15B);

FIG. 16A and FIG. 16B illustrate release of Texas Red molecules (MW=625.15) from thermosensitive liposome formulations over time at various temperatures. When approximately 40 mol % of unpolymerizable lipid (Compound B) was included in the thermosensitive liposomes, small MW molecules were significantly released at 42° C., but only marginally so at 37° C. (FIG. 16A). When approximately 40 mol % of unpolymerized lipid (Compound C) was included in the thermosensitive liposomes, small molecules were released at 45° C. but not at 42° C. or 37° C. (FIG. 16B);

FIG. 17 illustrates release of Texas Red molecules (MW=625.15) from thermosensitive liposome formulations over time at various temperatures. With approximately 60 mol % of unpolymerizable lipid (Compound C) included in the liposomes small MW molecules were released at 45° C. and 42° C., but not substantially so at 37° C.;

FIG. 18 illustrates release of Texas Red molecules (MW=625.15) from thermosensitive liposome formulations over time at various temperatures. With approximately 60 mol % of unpolymerizable lipid (Compound C) included in the liposomes small MW molecules were released at 45° C., and 42° C., but not substantially so at 37° C.;

FIG. 19 illustrates the percent release of the radioactive compound, ^{99m}Tc-HIDA, from thermosensitive liposome formulations incubated for 5 minutes (at either 37° C. or 47° C.) that were formulated with varying percentage of unpolymerizable lipid (Compound F). With successively higher content of unpolymerizable lipid, the liposomes became less stable and more of the active ingredient was released at 47° C., and even to some degree, at 37° C.; and

FIG. 20 illustrates the relative release of the radioactive compound, ^{99m}Tc-HIDA, from thermosensitive liposome formulations incubated for 5 minutes (at either 37° C. or 47° C.) that were formulated with varying percentage of unpolymerizable lipid (Compound C). With successively higher content of unpolymerizable lipid, the liposomes became less stable and more of the active ingredient was released at 47° C., and even to some degree, at 37° C.

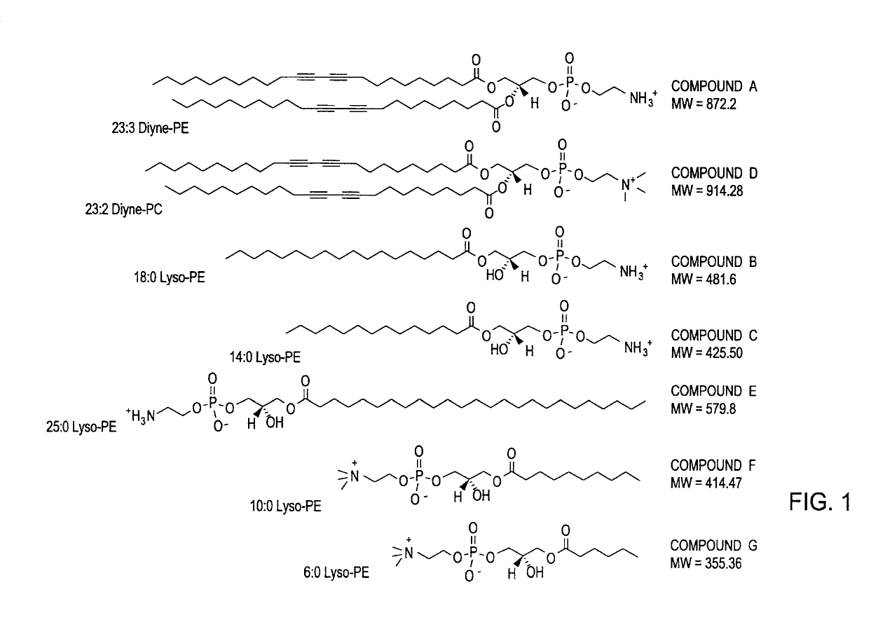

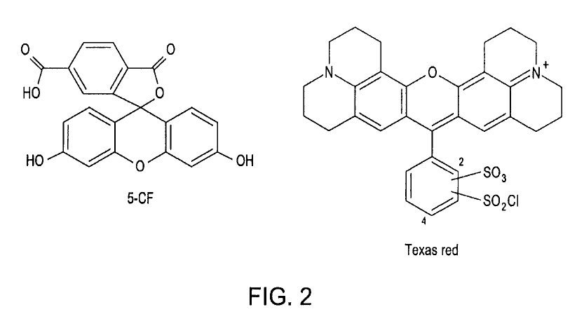

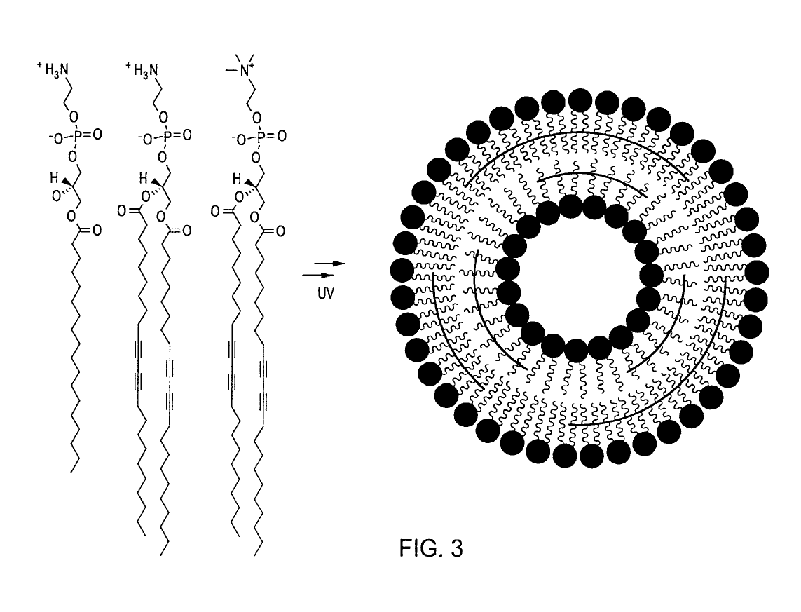

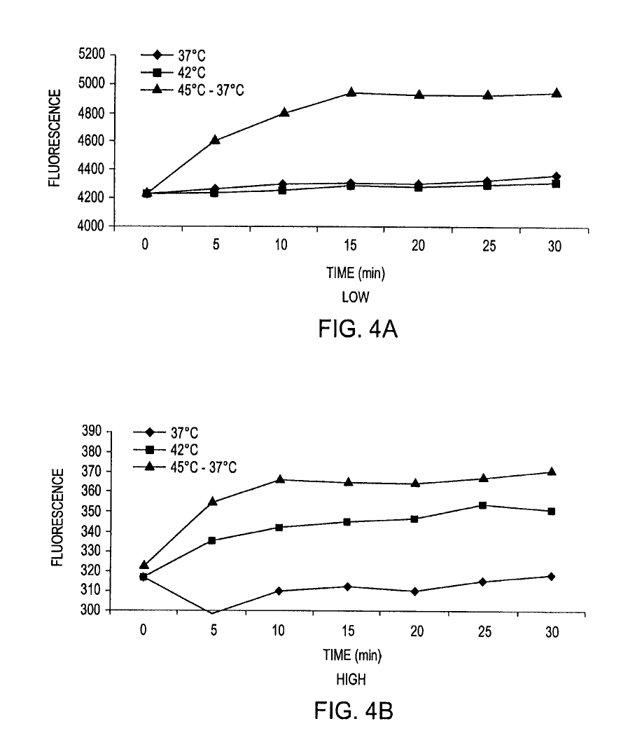

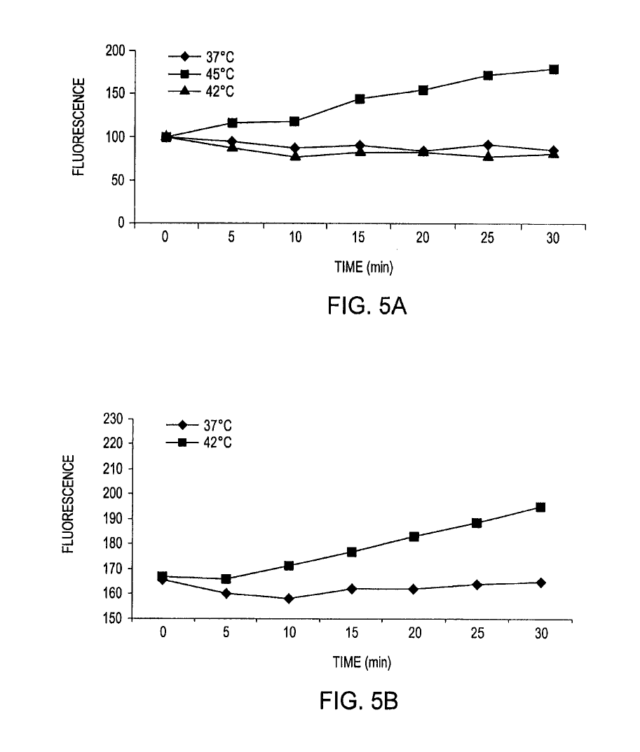

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure.

As described herein, the liposome compositions of the invention can be formed by any conventional means, including without limitation, by extrusion, sonication, or mechanical shearing, or a combination thereof. Entrapment of the active ingredient(s) into the lipid vesicles may also be accomplished by any conventional means, including exposure of the lipid vesicles to UV light in the presence of the active ingredient(s).

### Liposomes

Liposomes are microscopic vesicles, generally spherically-shaped, that are formed from one or more lipid bilayers. These bilayers are generally prepared from lipid molecules, which have the tendency to both form bilayers and minimize their surface area. The lipid molecules that make up a liposome have both hydrophilic and lipophilic (i.e., hydrophobic) portions.

Upon exposure to water, the lipid molecules form a bilayer membrane wherein the lipid ends of the molecules in each layer are directed to the center of the membrane, and the opposing polar ends form the respective inner and outer surfaces of the bilayer membrane. Thus, each side of the membrane presents a hydrophilic surface while the interior of the membrane includes a lipophilic medium (see e.g., U.S. Pat. No. 6,355,267, specifically incorporated herein in its entirety by express reference thereto).

Liposomes are useful in drug delivery due to their unique properties. A liposome encapsulates a region on aqueous solution inside a hydrophobic membrane; dissolved hydrophilic solutes cannot readily pass through the lipids. Hydrophobic chemicals can be dissolved into the membrane, and in this way, the liposomes can include both hydrophobic molecules and hydrophilic molecules. To deliver the molecules to sites of action, the lipid bilayer can fuse with other bilayers such as the cell membrane, thus delivering the liposome contents.

Liposomes according to the present invention may be prepared by any of a variety of techniques that are known in the art (see, e.g., U.S. Pat. No. 4,235,871, which is specifically incorporated herein in its entirety by express reference thereto). Entrapment of one or more active ingredient(s) or agent(s) within liposomes of the present invention may also be carried out using any conventional method in the art. In preparing liposome compositions of the present invention, stabilizers such as antioxidants and other additives may be used as long as they do not interfere with the purpose of the invention.

The fate and disposition of systemically injected liposomes depend on their physical properties, such as size, fluidity and surface charge. They may persist in tissues for hours or days, depending on their composition, and half-lives in the blood range from several minutes to several hours. Larger liposomes, such as MLVs and LUVs, are taken up rapidly by phagocytic cells of the reticuloendothelial system, but physiology of the circulatory system restrains the exit of such large species at most sites. They can exit only in places where large openings or pores exist in the capillary endothelium, such as the sinusoids of the liver or spleen. Thus, these organs are the predominate site of uptake. On the other hand, SUVs show a broader tissue distribution but still are sequestered highly in the liver and spleen. In general, this in vivo behavior limits the potential targeting of liposomes to only those organs and tissues accessible to their large size. These include the blood, liver, spleen, bone marrow and lymphoid organs.

Targeting is generally not a limitation in terms of the present invention. However, should specific targeting be desired, methods are available for this to be accomplished. Antibodies may be used to bind to the liposome surface and to direct the antibody and its drug contents to specific antigenic receptors located on a particular cell-type surface. Carbohydrate determinants (glycoprotein or glycolipid cell-surface components that play a role in cell-cell recognition, interaction and adhesion) may also be used as recognition sites as they have potential in directing liposomes to particular cell types. Mostly, it is contemplated that intravenous injection of liposomal preparations would be used, but other routes of administration are also conceivable as discussed supra.

### Exemplary Lipids for Formulation of Liposomes

The liposomes of the present invention can be prepared by any conventional technique(s), known to those of ordinary skill in the art, including for example extrusion, sonication, and such like, or a combination thereof. For example, liposomes can be readily formed by placing lipids in an aqueous solution and agitating the solution for a period of several seconds to several hours. The procedure spontaneously yields large, multilamellar liposomes or vesicles with diameters in the range of about 1 to 10 micrometers. These liposomes include from two to several hundred concentric lipid bilayers that may alternate with layers of the aqueous phase in which the lipids were present.

The thickness of the aqueous layer, and thus the total amount of aqueous phase trapped within the liposome, depends on the balance of electrostatic repulsion forces between charged lipids and Van der Waals attractive forces between bilayers as a whole. Thus, the aqueous spacing (and hence the volume of aqueous material trapped) increases with increasing proportion of charged lipids in the membrane and with decreasing concentrations of electrolytes (charged ions) in the aqueous phase.

Liposomes of various sizes may be employed. Small liposomes or vesicles formed are unilamellar and have a size in the range of about 20 to 400 nanometers, and can be produced by, for example without limitation, subjecting multi-lamellar vesicles to ultrasound, by extrusion under pressure through membranes having pores of defined size, or by high-pressure homogenization.

Exemplary lipids used in the formulation of the disclosed liposomes and liposomal particles preferably include, but are not limited to, phospholipids such as phosphatidylcholine, phosphatidylserine, phosphatidylglycerol, phosphatidylethanolamine, phosphatidylinositol and their mixtures, either alone, or in combination with one or more sphingolipids, glycolipids, fatty acids, cholesterols or cholesterol esters, or any combination thereof. Commercially-available lipids for use in the manufacture of liposomes include, without limitation, 1,2-diacyl-3-trimethylammonium-propanes (including, e.g. without limitation, 1,2-dioleoyl-3-trimethyl ammonium propane [DOTAP]); dimethyldioctadecyl ammonium bromide (DDAB); N-[1-(2,3-Dioloyloxy)propyl]-N,N,N-trimethylammonium methylsulfate; dimyristoyl; dipalmitoyl; distearoyl; 1,2-diacyl-3-dimethylammonium-propanes, (including, without limitation, dioleoyl, dimyristoyl, dipalmitoyl, and distearoyl); N-[1-[2,3-bis(oleoyloxy)]propyl]-N,N,N-trimethylammonium chloride (DOTMA); dioctadecylamidoglycylspermine (DOGS); 3β-[N—(N′,N′-dimethylaminoethane)carbamoyl]cholesterol; 2,3-dioleoyloxy-N-(2(sperminecarboxamido)-ethyl)-N,N-dimethyl-1-propanaminium trifluoroacetate (DOSPA); 1,2-diacyl-sn-glycero-3-ethylphosphocholines (including, without limitation, dioleoyl (DOEPC), dilauroyl, dimyristoyl, dipalmitoyl, distearoyl, palmitoyl-oleoyl); β-alanyl cholesterol; cetyl trimethyl ammonium bromide (CTAB); N-t-butyl-N′-tetradecyl-3-tetradecylamino propionamidine (diC14-amidine); O,O′-ditetradecanolyl-N-(trimethylammonioacetyl) diethanolamine chloride (14Dea2); 1,3-dioleoyloxy-2-(6-carboxy-spermyl)-propylamide (DOSPER); N,N,N′,N′-tetramethyl-N,N-bis(2-hydroxylethyl)-2,3-dioleoyloxy-1,4-butanediammonium iodide; 1-[2-acyloxy)ethyl]2-alkyl (alkenyl)-3-(2-hydroxyethyl)imidazolinium chloride derivatives such as 1-[2-(9(Z)-octadecenoyloxy)ethyl]-2-(8(Z)-heptadecenyl-3-(2-hydroxyethyl)-imidazolinium chloride (DOTIM), 1-[2-(hexadecanoyloxy)ethyl]-2-pentadecyl-3-(2-hydroxyethyl)imidazolinium chloride (DPTIM); 1-[2-tetradecanoyloxy)ethyl]-2-tridecyl-3-(2-hydroxyethyl)imidazolium chloride (DMTIM); 2,3-dialkyloxypropyl quaternary ammonium compound derivates, containing a hydroxyalkyl moiety on the quaternary amine, such as 1,2-dioleoyl-3-dimethyl-hydroxyethyl ammonium bromide (DORI); 1,2-dioleyloxypropyl-3-dimethyl-hydroxyethyl ammonium bromide (DORIE); 1,2-dioleyl-oxypropyl-3-dimethyl-hydroxypropyl ammonium bromide (DORIE-HP); 1,2-dioleyloxypropyl-3-dimethyl-hydroxybutyl ammonium bromide (DORIE-HB); 1,2-dioleyloxypropyl-3-dimethyl-hydroxypentyl ammonium bromide (DORIE-HPe); 1,2-dimyristyloxypropyl-3-dimethyl-hydroxylethyl ammonium bromide (DMRIE); 1,2-dipalmityloxypropyl-3-dimethyl-hydroxyethyl ammonium bromide (DPRIE); and 1,2-disteryloxypropyl-3-dimethyl-hydroxyethyl ammonium bromide (DSRIE) (for additional details see e.g., U.S. Pat. No. 7,112,338, specifically incorporated herein in its entirety by express reference thereto). Many of the above-mentioned lipids are available commercially from, e.g., Avanti Polar Lipids, Inc. (Alabaster, Ala., USA); Sigma Chemical Co. (St. Louis); Invitrogen Inc. (formerly Molecular Probes, Inc. Eugene, Oreg., USA); Northern Lipids, Inc. (Burnaby, BC, CANADA); Roche Molecular Biochemicals; Promega Corp. (Madison, Wis., USA); NDF Corp. (Tokyo, JAPAN); Soya Phospholipids, Inc. (Zhengzhou Siwei Grain & Oil Engineering & Technology Co., Ltd., Zhengzhou, CHINA); BioMol. Intl. Inc. (Plymouth Meeting, Pa., USA); Matreya Biochemicals LLC., (Pleasant Gap, Pa., USA); PhosphoTech Laboratories, (Saint-Herblain Cedex, FRANCE).

In various embodiments, one or more amphipathic lipids may be employed. “Amphipathic lipids” refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include, but are not limited to, sphingomyelin, phosphatidylcholine, phosphatidylethanolamine, phosphatidylserine, phosphatidylinositol, phosphatidic acid, palm itoyloleoyl phosphatdylcholine, lysophosphatidylcholine, lysophosphatidylethanolamine, dipalmitoylphosphatidylcholine, dioleoylphosphatidylcholine, distearoylphosphatidylcholine, dilinoleoylphosphatidylcholine, or any combination thereof.

Other phosphorus-lacking compounds, such as sphingolipids, glycosphingolipid families, diacylglycerols, and β-acyloxyacids may also be used in preparation of the disclosed liposome compositions, and such compositions may also readily be mixed with other lipids, including, without limitation, such as triglycerides, sterols, and the like, including any combinations thereof.

In some embodiments, the liposomes are also fusogenic or have a fusogenic coating. Fusogenic liposomes and coatings are known to those of ordinary skill in the art and are exemplified, for example, in U.S. Pat. No. 5,885,613, specifically incorporated herein in its entirety by express reference thereto.

In certain embodiments, it may be desirable to target the liposome compositions of the present invention using targeting moieties that are specific to a cell type or tissue. Targeting of liposomes using a variety of targeting moieties, such as ligands, cell surface receptors, glycoproteins, vitamins (e.g., riboflavin) and monoclonal antibodies, has been previously described (see, e.g., U.S. Pat. Nos. 4,957,773 and 4,603,044, each of which is specifically incorporated herein in its entirety by express reference thereto).

In exemplary embodiments, the polymerizable lipid component includes 23:2 1,2-bis(10,12-tricosadiynoyl)-sn-glycero-3-phosphoethanolamine (23:2 diyne-phosphatidyl ethanolamine; diyne-PE), and the unpolymerizable lipid component includes 18:0 1-stearoyl-2-hydroxy-sn-glycero-3-phosphoethanolamine (18:0 lyso-PE). In the practice of the invention, it is also contemplated that these two components may be formulated into liposomes that include approximately 60 mol % to 85 mol % of the 23:2 diyne-PE, and approximately 15 mol % to 40 mol % of 18:0 lyso-PE.

### Exemplary Delivered Agents

As used herein, an active agent “in the interior” or “entrapped within” the liposome is that which contained in the interior space of the liposome, compared to that partitioned into the lipid bilayer and contained within the vesicle membrane itself. As used herein, an active agent “within” or “entrapped within” the lipid bilayer of a liposome is carried as a part of the lipid bilayer, as opposed to being contained in the interior space of the liposome.

Agents that may be delivered to an animal or to a selected site within an animal using the liposomal composition of the present invention include, but are not limited to, therapeutic drugs, pharmacologic active agents, small molecules, nutraceuticals, hormones, stem cells, vitamins, cofactors, antibodies, antigens, antigen binding domains, enzymes, polynucleotides, proteins, peptides, nucleic acids, including DNA and RNA, peptide nucleic acids, steroids, ribozymes and other catalytic RNA molecules, antisense oligo- and poly-nucleotides, diagnostic agents, image contrast agents, radio-isotopes, diagnostic markers, antioxidants, chelators, ionophores, fluorophores, and any other molecules that is desired to be delivered to a particular anatomical or physiological site. Delivery may be targeted to one or more cell types, tissue types, organs, or systems within the body of the individual receiving the formulation.

Therapeutic agents include, without limitation, one or more of antibiotics, antimicrobials, antifungals, antihelminths, antivirals, antimycotics, antiamebics, anti-inflammatory agents, anti-neoplastic agents, including antitumor agents, antimitotics, antimetabolites, mutagens, alkylating agents, immunosuppressive agents, and the like, and any combination thereof.

Exemplary antineoplastic agents include, without limitation, one or more of anthracycline antibiotics (including, for example, without limitation, doxorubicin, daunorubicin, carcinomycin, N-acetyladriamycin, rubidazone, 5-imidodaunomycin, N-acetyl daunomycin, epirubicin, and the like), plant alkaloids (including, for example, without limitation, vincristine, vinblastine, etoposide, ellipticine, camptothecin, and the like). Mono- and di-terpenes (including, without limitation, paclitaxel, docetaxol (taxotere) and the like); mitotane, cisplatin, phenesterine, and any combination thereof.

Anti-inflammatory therapeutic agents suitable for use in the present invention include, without limitation, one or more of steroids and non-steroidal anti-inflammatory compounds (including, without limitation, prednisone, methyl-prednisolone, paramethazone, 11-fludrocortisol, triamciniolone, betamethasone and dexamethasone, ibuprofen, piroxicam, beclomethasone; methotrexate, azaribine, etretinate, anthralin, psoralins and the like); salicylates such as aspirin; and immunosuppresant agents (including, without limitation, cyclosporine and the like), and any combination thereof.

Additional pharmacological agents suitable for use in liposomes of the present invention include, without limitation, one or more of anesthetics (including, without limitation, methoxyflurane, isoflurane, enflurane, halothane, benzocaine, lidocane, bupivocane, ropivicane, and the like); antiulceratives (including, without limitation, cimetidine, famotidine, ranitidine and the like); antiseizure medications (including, without limitation, as barbituates; azothioprine (an immunosuppressant and antirheumatic agent); and muscle relaxants (such as dantrolene and diazepam), and any combination thereof.

Imaging agents suitable for use in the present liposome preparations include, without limitation, one or more of ultrasound contrast agents, radiocontrast agents (including, without limitation, radioisotopes or compounds containing radioisotopes, iodo-octanes, halocarbons, renografin, and the like), or magnetic contrast agents (including, without limitation, paramagnetic compounds and the like), and any combination thereof.

### Therapeutic and Diagnostic Kits

Kits including one or more of the disclosed liposome compositions or pharmaceutical formulations including such; and instructions for using the kit in a therapeutic, diagnostic, and/or other clinical embodiment also represent preferred aspects of the present disclosure. Such kits may further include one or more of the disclosed liposomal-vectored therapeutic or diagnostic reagents, either alone, or in combination with one or more additional therapeutic compounds, pharmaceuticals, and such like. The kits of the invention may be packaged for commercial distribution, and may further optionally include one or more delivery devices for the composition(s) to an animal (e.g., syringes, injectables, and the like). Such kits may be therapeutic kits for treating, preventing, or ameliorating the symptoms of a disease, deficiency, dysfunction, and/or injury, and may include one or more of the thermally-activatable liposome compositions, and instructions for using the kit in a therapeutic and/or diagnostic medical regimen.

The container for such kits may typically include at least one vial, test tube, flask, bottle, syringe or other container means, into which the liposome composition(s) may be placed, and preferably suitably aliquotted. Where a second liposome composition or a second delivered agent is also provided, the kit may also contain a second distinct container into which this second composition may be placed. Alternatively, the plurality of liposomal-based compositions may be prepared in a single pharmaceutical composition, and may be packaged in a single container, such as a vial, flask, syringe, catheter, cannula, bottle, or other suitable single container.

The kits of the present invention may also typically include a retention mechanism adapted to contain or retain the vial(s) or other container(s) in close confinement for commercial sale, such as, e.g., injection or blow-molded plastic containers into which the desired vial(s) or other container(s) may be retained to minimize or prevent breakage, exposure to sunlight, or other undesirable factors, or to permit ready use of the composition(s) included within the kit.

Alternatively, for the preparation of diagnostic kits, and for methods relating to the use of liposomal-vectored diagnostic compounds, such kits may be prepared that include at least one liposomal formulation as disclosed herein and instructions for using the composition in diagnosis. The container for such kits may typically include at least one vial, test tube, microcentrifuge tube, or other container, into which the antisense composition(s) may be placed and suitably aliquotted. Where a radiolabel or fluorogenic label or other such detecting component is included within the kit, the labeling agent may be provided either in the same container as the liposomal composition, or may alternatively be placed in a second distinct container into which this second composition may be placed and suitably aliquotted. Alternatively, the diagnostic liposomal-vectored compositions may be prepared in combination with one or more additional reagents in a single container, and in most cases, the kit will also typically include a retention mechanism adapted to retain or contain the vial(s) or other container(s) in close confinement for commercial sale and/or convenient packaging and delivery to minimize or avoid any undesirable environmental factors.

In particular, such kits may comprise one or more of the disclosed thermosensitive liposomal compositions in combination with instructions for using the composition in the diagnosis, prophylaxis, or treatment of a mammal, and may typically further include containers prepared for convenient commercial packaging.

As such, preferred animals for administration of the pharmaceutical compositions disclosed herein include mammals, and particularly humans. Other preferred animals include murines, bovines, equines, porcines, canines, and felines. The composition may include partially or significantly purified liposomal-based diagnostic or therapeutic compositions, either alone, or in combination with one or more additional active ingredients, diagnostics, pharmaceuticals, or therapeutic agents, including, without limitation, those compounds that may be obtained from natural or recombinant sources, or which may be obtainable naturally or either chemically synthesized, or alternatively produced in vivo or in vitro from one or more populations of host cells (including, without limitation, recombinant cells lines and the like that express one or more RNA or DNA segments that encode an active ingredient(s) in accordance with the present invention.

Therapeutic kits may also be prepared that comprise at least one of the liposomal carrier molecules disclosed herein and instructions for using compositions and formulations including them in the diagnosis, treatment, therapy, or amelioration of symptoms of a selected patient. The container means for such kits may typically comprise at least one vial, test tube, flask, bottle, syringe or other container means, into which the disclosed liposomal composition(s) may be placed, and preferably suitably aliquotted. Where a second diagnostic or therapeutic composition is also provided, the kit may also contain a second distinct container means into which this second composition may be placed. Alternatively, the compositions of the present invention may be prepared in a single pharmaceutical composition, and may be packaged in a single container means, such as a vial, flask, syringe, bottle, or other suitable single container means. The kits of the present invention will also typically include a means for containing the vial(s) in close confinement for commercial sale, such as, e.g., injection or blow-molded plastic containers into which the desired vial(s) are retained.

### Pharmaceutical Compositions

The liposomal formulations and drug-delivery vehicles of the present invention may be prepared in a variety of compositions, and may also be formulated in appropriate pharmaceutical carriers for administration to human and/or animal subjects. The liposomal formulations of the present invention and compositions comprising them provide new and useful diagnostics and therapeutics for the diagnosis, identification, treatment, control, and/or amelioration of symptoms of a variety of diseases, conditions, disorders, dysfunctions, and such like. Moreover, pharmaceutical compositions comprising one or more of the liposomal formulations disclosed herein, provide significant advantages over existing conventional therapies—namely, (1) their reduced side effects, (2) their increased efficacy for prolonged periods of time, (3) their ability to be precisely formulated for controlled delivery of substances to affected individuals; and (4) their ability to be targeted to one or more particular regions of the body by application of external heat, high-intensity focused ultrasound (HIFU) energy, and the like.

The invention also provides compositions comprising one or more of the disclosed liposomal delivery vehicles formulated for administration to an animal or a population of animal cells, tissues, organs, and such like. As described hereinbelow, such compositions may further comprise a pharmaceutical excipient, buffer, or diluent, and may be formulated for administration to an animal, and particularly a human being. Such compositions may further optionally comprise a microsphere, a microparticle, a nanosphere, or a nanoparticle, or may be otherwise formulated for administration to the cells, tissues, organs, or body of a mammal in need thereof. Such compositions may be formulated for use in therapy, such as for example, in the amelioration, prevention, or treatment of one or more conditions such as peptide deficiency, polypeptide deficiency, tumor, cancer or other malignant growth, neurological dysfunction, autoimmune diseases, lupus, cardiovascular disease, pulmonary disease, ischemia, stroke, cerebrovascular accidents, diabetes and diseases of the pancreas, neural diseases, including Alzheimer's, Huntington's, Tay-Sach's, and Parkinson's diseases, memory loss, trauma, motor impairment, and the like, as well as biliary, renal or hepatic disease or dysfunction, as well as musculoskeletal diseases including, for example, without limitation, arthritis, cystic fibrosis (CF), amyotrophic lateral sclerosis (ALS), multiple sclerosis (MS), muscular dystrophy (MD), and such like, to name only a few.

In certain embodiments, the present invention concerns formulation of one or more of the liposomal drug delivery vehicles compositions disclosed herein in pharmaceutically acceptable solutions for administration to a cell or an animal, either alone or in combination with one or more other modalities of diagnosis, detection, therapy, and in particular, for diagnosis or therapy of one or more human cells, tissues, organs, and in the amelioration of symptoms of disorders, diseases, conditions, trauma, or dysfunctions affecting man.

Formulation of pharmaceutically-acceptable excipients and carrier solutions is well-known to those of skill in the art, as is the development of suitable dosing and treatment regimens for using the particular compositions described herein in a variety of treatment regimens, including e.g., without limitation, oral, parenteral, intravenous, intranasal, intratumoral, and intramuscular routes of administration.

Typically, the thermosensitive liposomal drug delivery vehicles of the present invention may be formulated to contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound(s) in each diagnostically- or therapeutically-useful composition may be prepared is such a way that a suitable dosage of the diagnostic or therapeutic agent will be obtained in any given unit dose of the liposomal carrier molecules disclosed herein. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the liposomal-based diagnostic and/or therapeutic components in suitably formulated pharmaceutical compositions disclosed herein either subcutaneously, intraocularly, intravitreally, parenterally, intravenously, intracerebroventricularly, intramuscularly, intrathecally, orally, intraperitoneally, by oral or nasal inhalation, or by direct injection to one or more neural cells, nervous tissues, or even by direct injection or administration to the brain, CNS or to the peripheral nervous system. The methods of administration may also include those modalities as described in U.S. Pat. Nos. 5,543,158; 5,641,515 and 5,399,363 (each of which is specifically incorporated herein in its entirety by express reference thereto). Solutions of the active compounds as freebase or pharmacologically acceptable salts may be prepared in sterile water and may also suitably mixed with one or more surfactants, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms of the thermosensitive liposomal-based delivery compositions suitable for injectable use include sterile aqueous solutions or dispersions and sterile powders for the extemporaneous preparation of sterile injectable solutions or dispersions (see e.g., U.S. Pat. No. 5,466,468, specifically incorporated herein in its entirety by express reference thereto). In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the thermosensitive partially polymerized liposomal formulations in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may also be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

The particular amount of compositions employed, and the particular time of administration, or dosage regimen for compositions employing the disclosed liposomal vehicles will be within the purview of a person of ordinary skill in the art having benefit of the present teaching. It is likely, however, that the administration of diagnostically- or therapeutically-effective amounts of the disclosed liposomal-based carrier molecules may be achieved by a single administration, to provide the desired diagnostic or therapeutic benefit to the patient undergoing such treatment. Alternatively, in some circumstances, it may be desirable to provide multiple, or successive administrations of the disclosed compositions, either over a relatively short, or a relatively prolonged period of time, as may be determined by the medical practitioner overseeing the administration of such compositions. For example, the compositions of the present invention may be administered to a subject as a single dose, or divided into two or more administrations as may be required to achieve diagnosis, detection, therapy, or amelioration of one or more symptoms of the particular disease, condition, trauma, dysfunction, or disorder being diagnosed and/or treated.

### Exemplary Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of clarity, the following specific terms are defined below:

As used herein, the term “comprising” and its cognates are used in their inclusive sense; that is, equivalent to the term “including” and its corresponding cognates.

In accordance with long standing patent law convention, the words “a” and “an” when used in this application, including the claims, denotes “one or more.”

The term “e.g.,” as used herein, is used merely by way of example, without limitation intended, and should not be construed as referring only those items explicitly enumerated in the specification.

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

As used herein, the term “patient” (also interchangeably referred to as “recipient” “host” or “subject”) refers to any host that can serve as a recipient for one or more of the vascular access devices as discussed herein. In certain aspects, the recipient will be a vertebrate animal, which is intended to denote any animal species (and preferably, a mammalian species such as a human being). In certain embodiments, a “patient” refers to any animal host, including but not limited to, human and non-human primates, avians, reptiles, amphibians, bovines, canines, caprines, cavines, corvines, epines, equines, felines, hircines, lapines, leporines, lupines, murines, ovines, porcines, racines, vulpines, and the like, including, without limitation, domesticated livestock, herding or migratory animals or birds, exotics or zoological specimens, as well as companion animals, pets, and any animal under the care of a veterinary practitioner.

As used herein, “carrier” includes any solvents, dispersion medium, vehicle, coating, diluent, buffer, antibacterial and antifungal agent, isotonic and absorption delaying agent, carrier solution, suspension, colloids, or such like. The use of such delivery media for pharmaceutically active substances is well known in the art. Except insofar as any conventional media or agent is incompatible with the active ingredient, its use in the therapeutic compositions is contemplated. Supplementary active ingredients can also be incorporated into one or more of the disclosed liposome compositions.

The term “liposomes” or “vesicles” or “liposome vesicles,” as used herein, means structures having lipid-containing membranes enclosing an aqueous interior. Structures having more than one layer of membranes are termed multilamellar vesicles (MLVs). Structures having one layer of membrane are termed unilamellar vesicles (ULVs). ULVs may be distinguished by their relative sizes as large or small ULVs (sometimes abbreviated “LUVs” and “SUVs,” respectively).

The term “large unilamellar vesicles” or “LUVs,” as used herein, means unilamellar vesicles having a diameter of about 100 nm to about 1.0 μm (1000 nm).

The term “critical micellar temperature” or “CMT” as used herein means the temperature above which the poloxamer molecules by themselves exist in aqueous medium as micells, and below which the poloxamer molecules by themselves exist as individual molecules (unimers) in solution.

The term “delivered agent” or “active ingredient” as used herein means any chemical compound that may be encapsulated within one or more liposomes or liposome particles for delivery to an animal or to one or more selected target site(s) within the body of the animal.

The terms “treatment,” “treating,” “treat” and the like are used herein to generally mean obtaining a desired pharmacologic and/or physiologic effect. The effect may be prophylactic in terms of completely, or partially, inhibiting or preventing a disease or symptom thereof and/or may be therapeutic in terms of a partial or complete stabilization or cure for a disease and/or adverse effect attributable to the disease.

As used herein, the term “hyperthermia” refers to the elevation of the temperature of a subject's body, or a part of a subject's body, compared to the normal temperature of the subject. Such elevation may be the result of a natural process (e.g., inflammation) or artificially induced (e.g., by the application of an external heat source or HIFU) for therapeutic or diagnostic purposes. As used herein, “hyperthermic administration” of an active agent refers to its administration in conjunction with the use of clinical hyperthermia in the subject at a preselected target site, to deliver a larger amount of active agent to the target site compared to that which would result from the administration of the active agent in the absence of hyperthermia.

The phrase “pharmaceutically-acceptable” refers to molecular entities and compositions that do not produce an allergic or similar untoward reaction when administered to a human, and in particular, when administered to the human eye. The preparation of an aqueous composition that contains a protein as an active ingredient is well understood in the art. Typically, such compositions are prepared as injectables, either as liquid solutions or as suspensions. Alternatively, they may be prepared in solid form suitable for solution in, or suspension in, liquid prior to injection. The liposome preparations may also be emulsified if needed.

As used herein, “pharmaceutically acceptable salt” refers to a salt that retains the desired biological activity of the parent compound and does not impart any undesired toxicological effects. Examples of such salts include, but are not limited to, acid addition salts formed with inorganic acids, for example, hydrochloric acid, hydrobromic acid, sulfuric acid, phosphoric acid, nitric acid, and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′-dibenzylethylenediamine or ethylenediamine; and combinations thereof.

The term “lipid” is used in its conventional sense as a generic term encompassing fats, lipids, and the alcohol-ether-soluble constituents of protoplasm, which are insoluble in water. The term encompasses both naturally occurring and synthetically produced lipids. Preferred lipids in connection with the present invention include, but are not limited to, cholesterol, phospholipids, including phophatidylcholines and phosphatidylethanolamines, and sphingomyelins, or any combination thereof.

The term “cationic lipid” is used herein to encompass any lipid of the invention (as defined above) which has a net positive charge at physiological pH.

The term “liposome” encompasses any compartment enclosed by a lipid bilayer. Liposomes are also referred to as lipid vesicles. In order to form a liposome the lipid molecules include elongated non-polar (hydrophobic) portions and polar (hydrophilic) portions.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example from a pH of about 5 to 7.

### EXAMPLES

The following examples are included to demonstrate illustrative embodiments of the invention. It should be appreciated by those of ordinary skill in the art that the techniques disclosed in the examples that follow represent techniques discovered by the inventor to function well in the practice of the invention, and thus can be considered to constitute preferred modes for its practice. However, those of ordinary skill in the art should, in light of the present disclosure, appreciate that many changes can be made in the specific embodiments which are disclosed and still obtain a like or similar result without departing from the spirit and scope of the invention.

### Example 1

### Preparation of Illustrative Liposome Compositions

Appropriate amounts of lipids were dissolved in chloroform or chloroform/methanol. The solvent was evaporated, and the residue was dried under vacuum while shielded from light. After the addition of fluorescent dye or radioactively-labeled molecules in PBS, the solution was sonicated to form a milky emulsion. The emulsion was transferred to a 10-mL Lipex extruder (Northern Lipids, Inc.) equipped with two stacked polycarbonate filter membranes having a pore size of 100 nm. The emulsion was then extruded a total of 11 times, then the solution was transferred onto Petri dishes maintained over ice, and irradiated at 254 nm for 1 hr with a Hoefer UVC 500 UVC crosslinker, yielding partially-polymerized liposomes. The liposomes were then purified with PD10 desalting columns and collection.

In an illustrative embodiment, a lipid composition was formulated to contain approximately 85% of 23:2 1,2-bis(10,12-tricosadiynoyl)-sn-glycero-3-phosphoethanolamine (23:2 diyne-PE; Avanti Polar Lipids, Inc. Catalog No.: 790145) and approximately 15% of 1-stearoyl-2-hydroxy-sn-glycero-3-phosphoethanolamine (18:0 lyso-PE; Avanti Polar Lipids, Inc. Catalog No.: 856715).

In another embodiment, a lipid composition was formulated to contain approximately 85% of 23:2 1,2-bis(10,12-tricosadiynoyl)-sn-glycero-3-phosphocholine (23:2 diyne-PC) and approximately 15% of 14:0 1,2-bis(10,12-tricosadiynoyl)-sn-glycero-3-phosphocholine (14:0 lyso-PE).

### Example 2

### Thermally-Activatable Partially-Polymerized Liposomes

The following example describes a liposome composition that includes a mixture of polymerizable and un-polymerizable lipids, wherein the majority of the lipids (greater than approximately 60%) are of the polymerizable variety. Without being bound by theory, since the polymerizable lipids will form cross-links after exposure to ultraviolet light, liposomal formulations of this type are believed to be relatively stable at normal human body temperature. When exposed to moderate heating (e.g., greater than about 39° C. or 40° C.), however, the lipids will at least partially phase separate, thereby releasing the encapsulated imaging, prophylactic, or therapeutic agent. With the proper lipid composition, substantially all partially-polymerized liposomes can be made to “open” with moderate heating (i.e., “leak” to release their contents) and yet close when exposed to normal body temperature (or lower).

In this example, the thermally-activatable partially-polymerized liposome formulation includes essentially of liposome forming lipids, the liposome forming lipids having active hydrophilic head groups formed to include one or more of diethylenetriaminepentaacetic acid, ethylenedinitriletetraacetic acid, tetraazacylododecane 1,4,7,10-tetraacetic acid and cyclohexane-1,2,-diamino-N,N-diacetate, greater than about 55% of the liposome-forming lipids having at least one hydrophobic tail group that contains a polymerizable functional group formed to include one or more of diacetylene, olefin, acetylene nitrile, styrene, ester, thiol, amide, α-unsaturated ketone, β-unsaturated ketone, α-unsaturated aldehyde, β-unsaturated aldehyde, and less that 45% of the liposome forming lipids having at least a first hydrophobic tail group that lacks a polymerizable functional group.

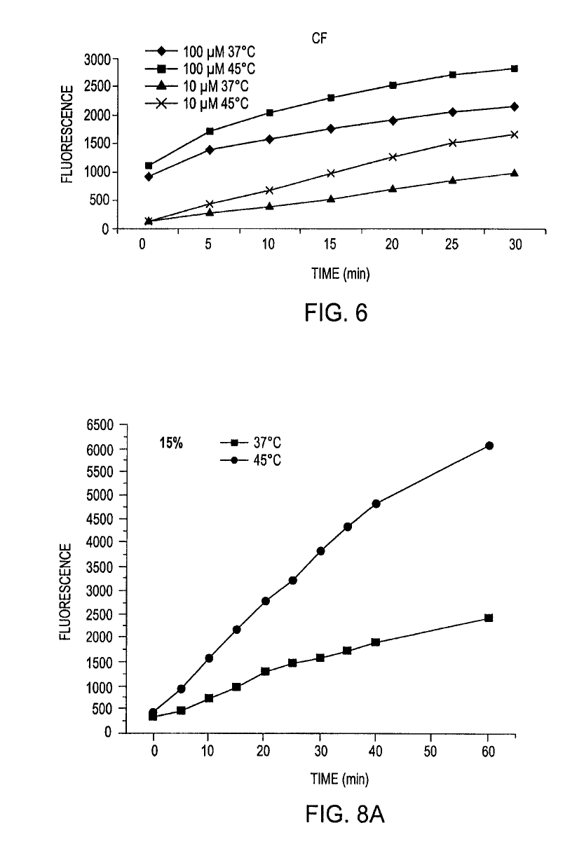

FIG. 6 illustrates results of release of Texas Red from liposome formulations over time at various temperatures. With approximately 50 mol % of unpolymerized lipids including the liposomes, the 3,000 MW molecule could be effectively released from the liposomes at 45° C. but not at lower temperatures including 42° C. and 37° C.

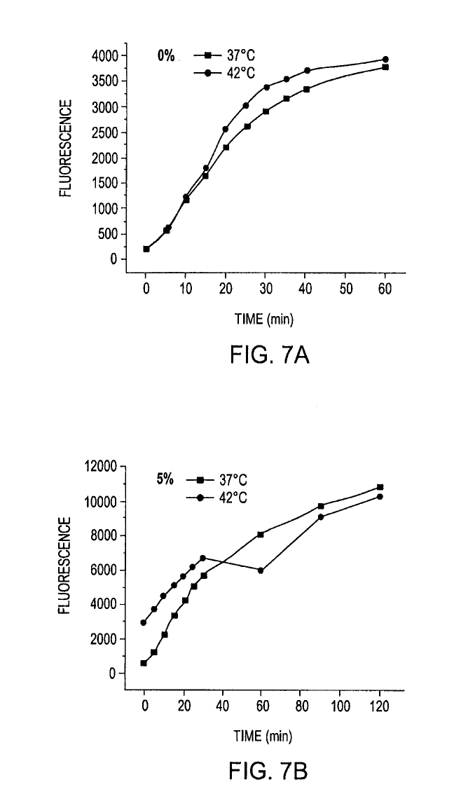

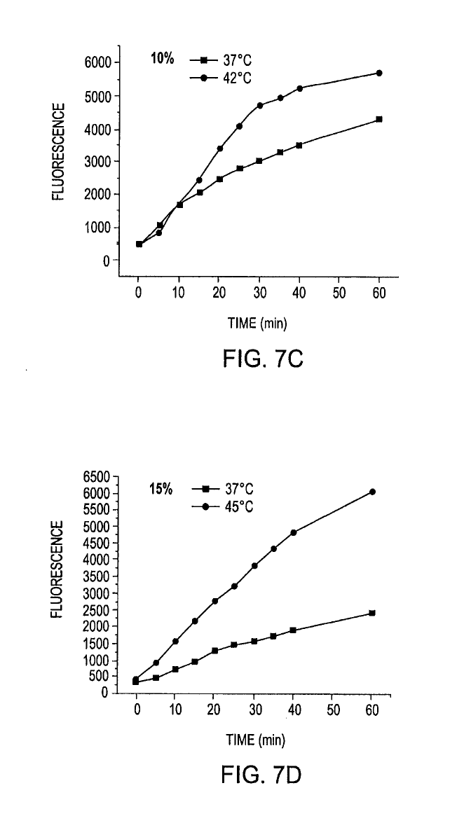

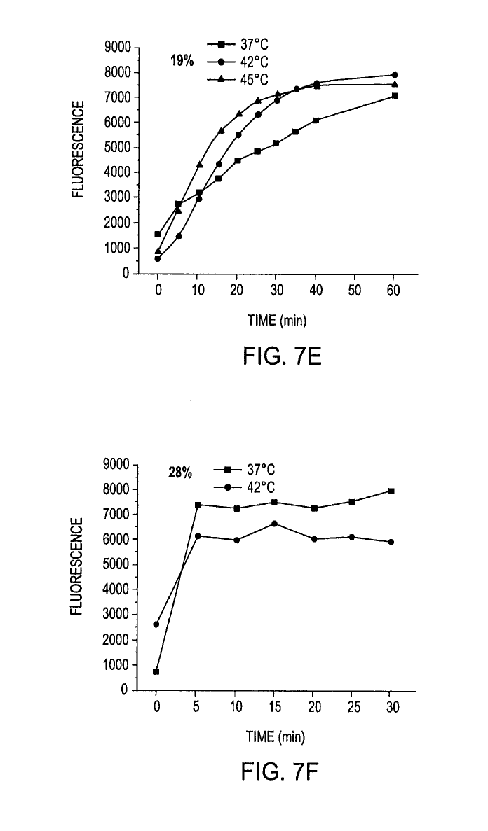

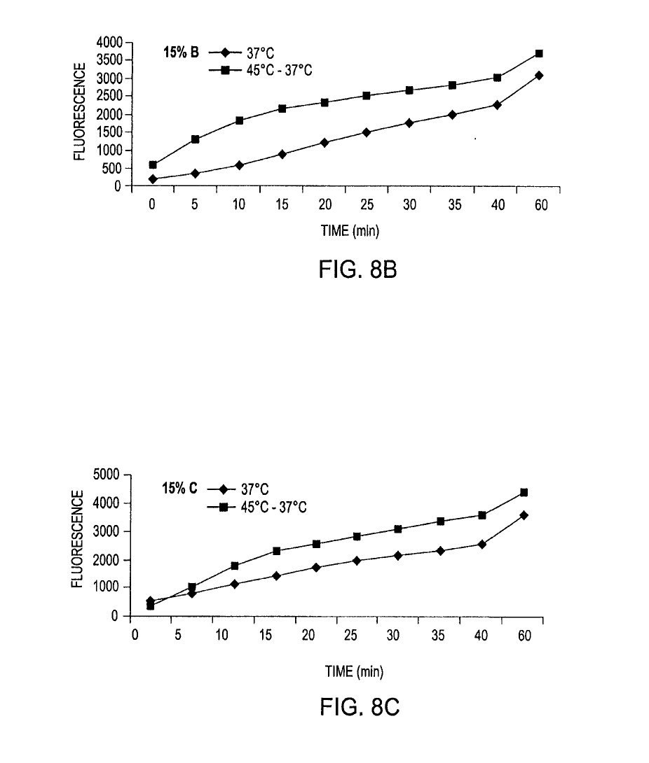

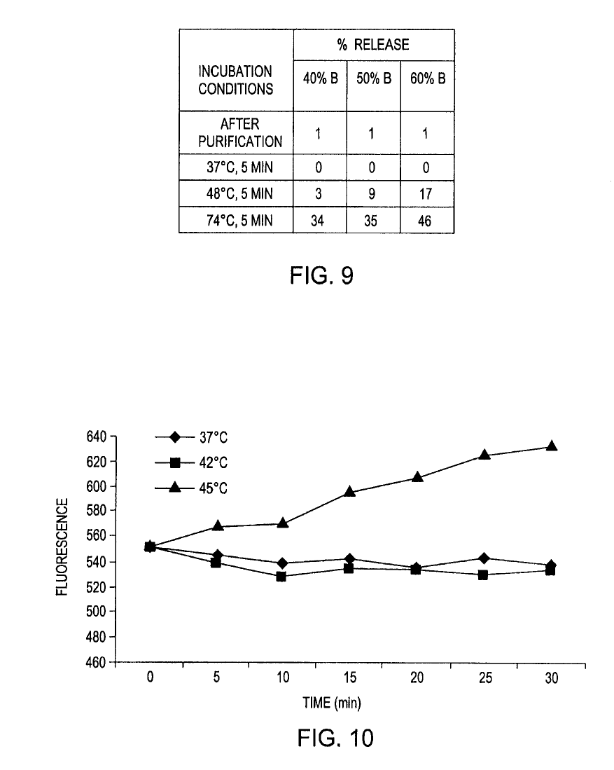

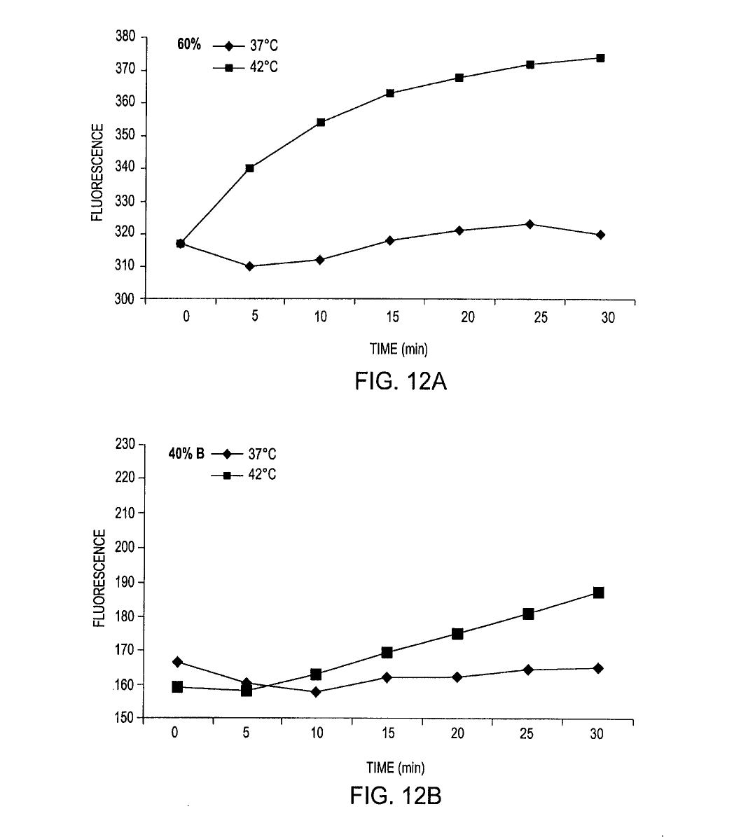

FIG. 7A-FIG. 7F and FIG. 12 illustrate the results of release of a small-molecular weight Texas Red (MW=625.15) from liposome formulations over time at various temperatures. When approximately 40 mol % of unpolymerized lipids were used to formulate the liposomes, the small MW molecule could be effectively released from the liposomes at 42° C. but not at 37° C. With no unpolymerized lipids were included in the liposome formulation, even small MW molecules (e.g., MW=625.15) were not effectively released at either 37° C. or 42° C. When approximately 60 mol % (FIG. 12A) of unpolymerized lipids were used to formulate the liposomes, the release of small MW molecules was much faster than from liposomes that were formulated with 40 mol % unpolymerized lipids (FIG. 12B).

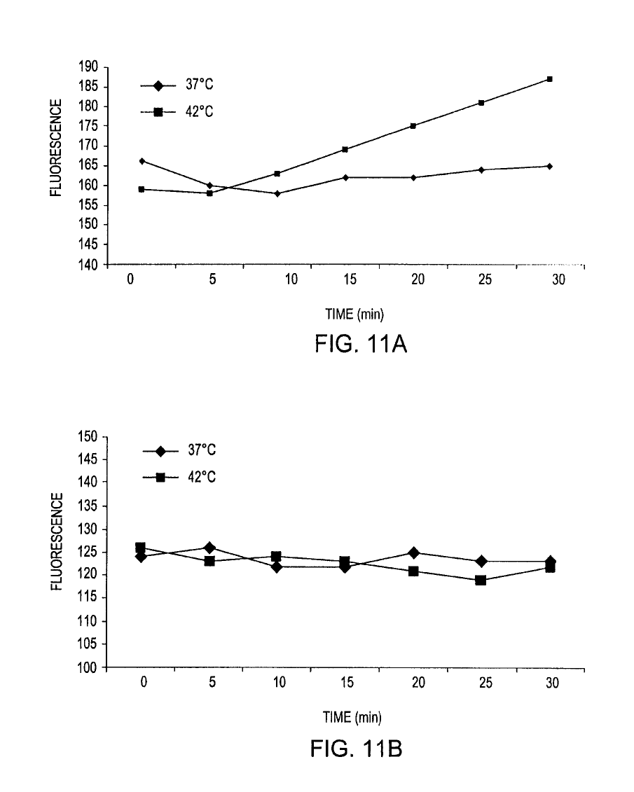

As shown in FIG. 10 when approximately 50 mol % of unpolymerizable lipids (Compound B) were included in the liposome formulations in accordance with one aspect of the invention, large MW molecules could be released at 45° C., but not at 42° C. or 37° C. Similarly, as shown in FIG. 11A and FIG. 11B, when approximately 40 mol % of unpolymerizable lipids (Compound B) were included in the liposome formulation small MW molecules were released at 42° C., but not at 37° C. When no unpolymerized lipids were present in the liposome formulation, even small molecules were not released at 42° C. or 37° C.

As illustrated in FIG. 12A and FIG. 12B when approximately 60 mol % of unpolymerizable lipids were included in the liposome formulation, the release of small MW molecules was much faster than when 40 mol % unpolymerizable lipids were used to formulate the liposomes.

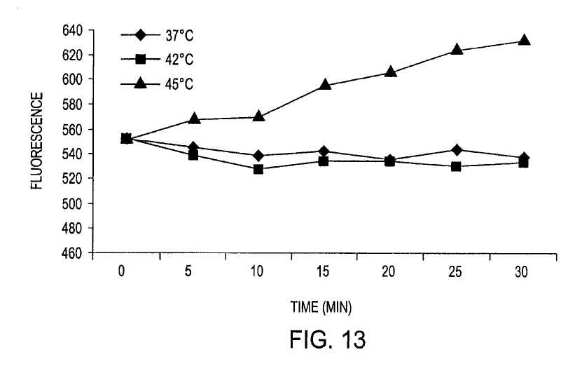

As shown in FIG. 13 when approximately 50 mol % of unpolymerizable lipids (Compound B) was included in the liposomal formulation, the release of large MW molecules was demonstrated at 45° C., but not at 42° C. or 37° C.

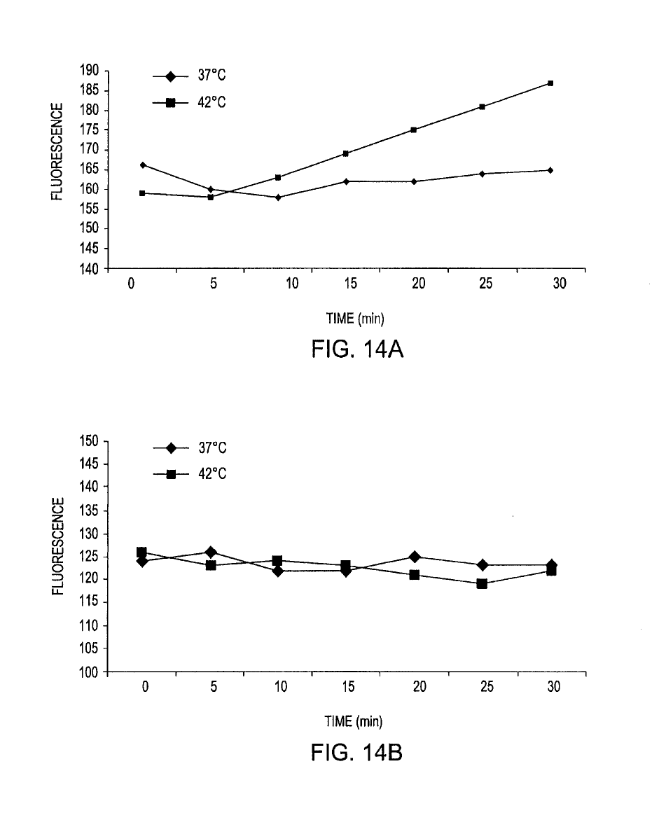

As shown in FIG. 14A and FIG. 14B, when approximately 40 mol % of unpolymerizable lipids (Compound B) was included in the liposomal formulation, (FIG. 14A), small-MW molecules were released at 42° C., but not at 37° C.; however, when fully polymerized lipids alone were used to form the liposome, small molecules were not released at either 42° C. or 37° C. (FIG. 14B).

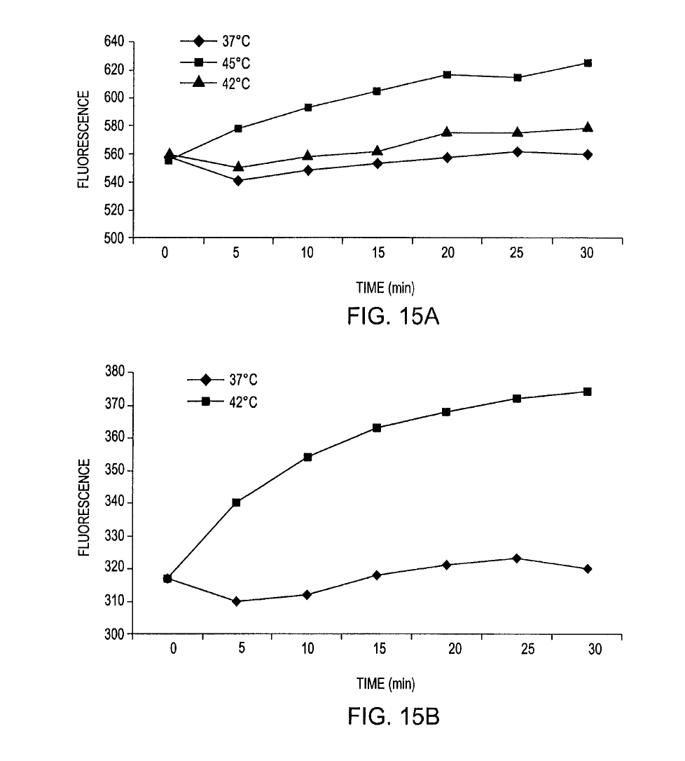

As illustrated in FIG. 15A and FIG. 15B, when approximately 37 mol % of unpolymerizable lipids (Compound B) were included in the liposomal formulation (FIG. 15A), small MW molecules were released at 45° C. and 42° C., but not at 37° C.; however, when 60 mol % of unpolymerized lipids (Compound B) were present in the liposome formulation, small molecules were released only at 42° C., and not at 37° C. (FIG. 15B).

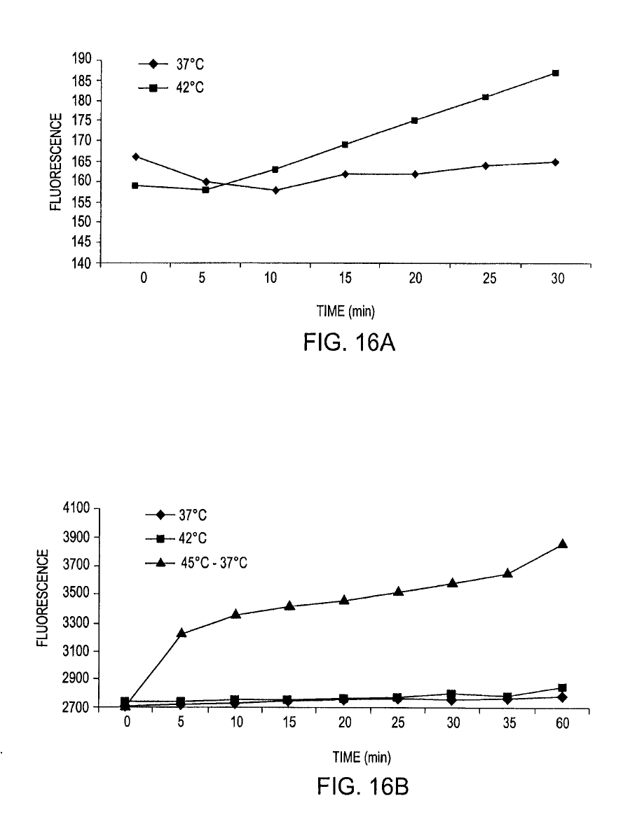

The results in FIG. 16A and FIG. 16B demonstrate that when approximately 40 mol % of unpolymerizable lipid (Compound B) was included in the thermosensitive liposomal formulation, small-MW molecules were significantly released at 42° C., but only marginally so at 37° C. (FIG. 16A). When approximately 40 mol % of unpolymerized lipid (Compound C) was included in the thermosensitive liposomes, small molecules were released at 45° C. but not at 42° C. or 37° C. (FIG. 16B);

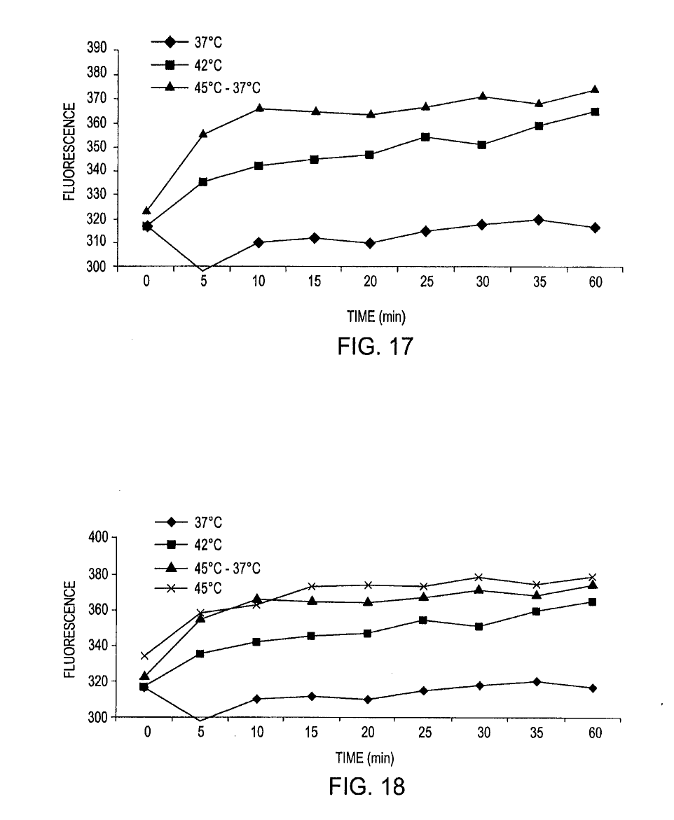

As shown in FIG. 17, with approximately 60 mol % of unpolymerizable lipid (Compound C) included in the liposomal formulation, small MW molecules were significantly released at 45° C. and 42° C., but not substantially so at 37° C. Likewise, as shown in FIG. 18, when approximately 60 mol % of unpolymerizable lipid (Compound C) was used to prepare the thermosensitive liposomes, small MW molecules were released at 45° C., and 42° C., but not substantially so at 37° C.

### Example 3

### Thermosensitive Liposomes Comprising Radioactive Compounds

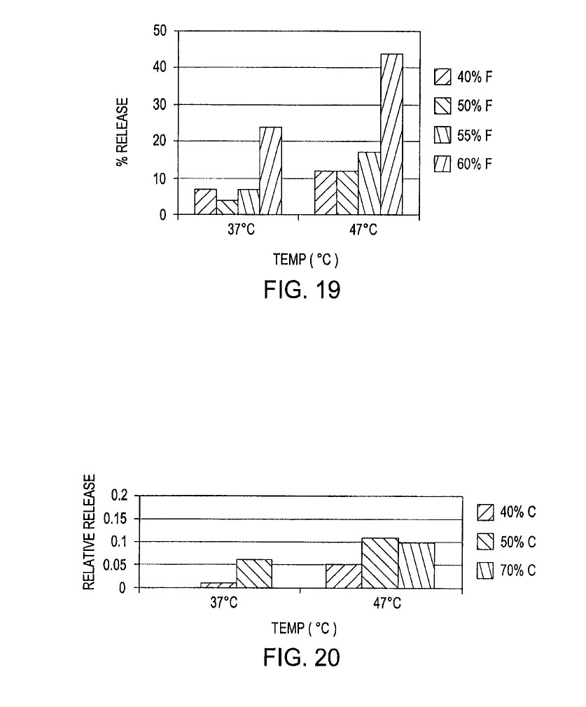

As shown in FIG. 19 when successively higher content of unpolymerizable lipid, the liposomes became less stable and more of the active ingredient was released at 47° C., and even to some degree, at 37° C. Similarly, in FIG. 20 it was shown that when successively higher content of unpolymerizable lipid was employed, the liposomes became less stable and more of the active ingredient was released at 47° C., and even to some degree, at 37° C.

### REFERENCES

The following references, to the extent that they provide exemplary procedural or other details supplementary to those set forth herein, are specifically incorporated herein in their entirety by express reference thereto:
Chaieb et al., “Glassy conformations in wrinkled membranes,” Phys. Rev. Lett., 96:078101, 2006.
Chaieb et al., “Why the wrinkling transition in partially polymerized membranes is not universal?” J. Theor. Biol., 251(1):60-67, 2008.
Snitko et al., “High-specificity of human secretory class II phospholipase A2 for phosphatidic acid,” Biochem. J., 321(3):737-41, 1997.
Takeoka et al., “Phase separation of polymerized mixed liposomes: analysis of release behavior of entrapped molecules with skeletonization,” Macromolecules, 24:1279-1283, 1991.
U.S. Pat. No. 7,393,478, entitled “Therapy for human cancers using cisplatin and other drugs or genes encapsulated into liposomes.”
U.S. Pat. No. 7,384,923, entitled “Liposomes.”
U.S. Pat. No. 7,381,421, entitled “Liposomes.”
U.S. Pat. No. 7,371,404, entitled “Amphoteric liposomes and their use.”
U.S. Pat. No. 7,354,567, entitled “Method of encapsulating metal complex within liposomes.”
U.S. Pat. No. 7,312,206, entitled “Sterol derivatives, liposomes comprising sterol derivatives and method for loading liposomes with active substances.”
U.S. Pat. No. 7,273,620, entitled “Triggered release of liposomal drugs following mixing of cationic and anionic liposomes.”
U.S. Pat. No. 7,262,173, entitled “Chemosensitizing with liposomes containing oligonucleotides.”
U.S. Pat. No. 7,205,273, entitled “Fusogenic liposomes.”
U.S. Pat. No. 7,153,933, entitled “Solid phase method for synthesis peptide-spacer-lipid conjugates, conjugates synthesized thereby and targeted liposomes containing the same.”
U.S. Pat. No. 7,153,490, entitled “Liposomes encapsulating anticancer drugs and use thereof in the treatment of malignant tumors.”
U.S. Pat. No. 7,150,883, entitled “Self forming, thermodynamically stable liposomes and their applications.”
U.S. Pat. No. 7,067,697, entitled “Cationic liposomes for gene transfer.”
U.S. Pat. No. 7,025,988, entitled “Liposomes.”
U.S. Pat. No. 7,002,042, entitled “Cationic liposomes for gene transfer.”
U.S. Pat. No. 6,989,153, entitled “Radiation sensitive liposomes.”
U.S. Pat. No. 6,964,778, entitled “Temperature controlled content release from liposomes.”
U.S. Pat. No. 6,958,160, entitled “Self forming, thermodynamically stable liposomes and their applications.”
U.S. Pat. No. 6,767,554, entitled “Use of complexes among cationic liposomes and polydeoxyribonucleotides and medicaments.”
U.S. Pat. No. 6,743,638, entitled “Detection system using liposomes and signal modification.”
U.S. Pat. No. 6,726,926, entitled “Gene-entrapped liposomes preparation and process for the preparation thereof.”
U.S. Pat. No. 6,726,925, entitled “Temperature-sensitive liposomal formulation.”
U.S. Pat. No. 6,690,976, entitled “Thermotherapy method for treatment and prevention of breast cancer and cancer in other organs.”
U.S. Pat. No. 6,656,498, entitled “Cationic liposomes for gene transfer.”
U.S. Pat. No. 6,623,430, entitled “Method and apparatus for safety delivering medicants to a region of tissue using imaging, therapy and temperature monitoring ultrasonic system.”
U.S. Pat. No. 6,610,322, entitled “Self forming, thermodynamically stable liposomes and their applications.”
U.S. Pat. No. 6,610,304, entitled “Liposomes containing multiple branch peptide constructions for use against human immunodeficiency virus.”
U.S. Pat. No. 6,596,543, entitled “Use of liposomes of defined composition and size for the preparation of prothrombin time reagents.”
U.S. Pat. No. 6,596,305, entitled “Method of controlling the size of liposomes.”
U.S. Pat. No. 6,593,294, entitled “Pharmaceutical composition comprising Factor VIII and neutral liposomes.”
U.S. Pat. No. 6,592,843, entitled “Radioactive therapeutic liposomes.”
U.S. Pat. No. 6,562,371, entitled “Liposomes.”
U.S. Pat. No. 6,511,677, entitled “Polymerizable fatty acids, phospholipids and polymerized liposomes therefrom.”
U.S. Pat. No. 6,511,676, entitled “Therapy for human cancers using cisplatin and other drugs or genes encapsulated into liposomes.”
U.S. Pat. No. 6,500,453, entitled “Polymerizable fatty acids, phospholipids and polymerized liposomes therefrom.”
U.S. Pat. No. 6,469,084, entitled “Process for preparing an aqueous composition in gel form and compositions obtainable from this process, especially a composition containing vesicles, in particular liposomes.”
U.S. Pat. No. 6,458,381, entitled “Lipids and their use, for example, in liposomes.”
U.S. Pat. No. 6,451,338, entitled “Liposomes containing particulate materials.”
U.S. Pat. No. 6,447,800, entitled “Method of loading preformed liposomes using ethanol.”
U.S. Pat. No. 6,426,086, entitled “pH-sensitive, serum-stable liposomes.”
U.S. Pat. No. 6,417,326, entitled “Fusogenic liposomes.”
U.S. Pat. No. 6,387,397, entitled “Polymerized liposomes targeted to M cells and useful for oral or mucosal drug delivery.”
U.S. Pat. No. 6,380,359, entitled “Liposomes comprising peptide antigens derived from X protein of hepatitis B virus.”
U.S. Pat. No. 6,133,451, entitled “Method of delivery of skin treatment agents using free-dried liposomes.”
U.S. Pat. No. 6,200,598, entitled “Temperature-sensitive liposomal formulation.”
U.S. Pat. No. 5,810,888, entitled “Thermodynamic adaptive phased array system for activating thermosensitive liposomes in targeted drug delivery.”
U.S. Pat. No. 5,720,976, entitled “Thermosensitive liposome and process for preparing the same.”
U.S. Pat. No. 5,540,936, entitled “Method of producing liposomes.”
U.S. Pat. No. 5,209,720, entitled “Methods for providing localized therapeutic heat to biological tissues and fluids using gas filled liposomes.”
U.S. Pat. No. 5,094,854, entitled “Liposome composition useful for hyperthermia therapy.”

All of the compositions and methods disclosed and claimed herein can be made and executed without undue experimentation in light of the present disclosure. While the compositions and methods of this invention have been described in terms of exemplary embodiments, it will be apparent to those of skill in the art that variations may be applied to the composition, methods and in the steps or in the sequence of steps of the method described herein without departing from the concept, spirit and scope of the invention. More specifically, it will be apparent that certain agents that are both chemically- and physiologically-related may be substituted for the agents described herein while the same or similar results would be achieved. All such similar substitutes and modifications apparent to those of ordinary skill in the art are deemed to be within the spirit, scope and concept of the invention as defined by the appended claims. Accordingly, the exclusive rights sought to be patented are as described in the claims below.

1. A pharmaceutical composition comprising a population of liposomes and one or more active ingredient(s) contained substantially within the population of liposomes, wherein the composition comprises from about 30 mole % to about 70 mole % of a first polymerizable lipid component, and from about 70 mole % to about 30 mole % of a first unpolymerizable lipid component, and wherein greater than about 40% of the contained active ingredient(s) is released from the population of liposomes in an environmental temperature of from about 39° C. to about 45° C.

2. The pharmaceutical composition of claim 1, wherein greater than about 50% of the contained active ingredient(s) is released from the population of liposomes in an environmental temperature of from about 39° C. to about 45° C.

3. The pharmaceutical composition of claim 1, wherein the composition comprises from about 40 mole % to about 60 mole % of a first polymerizable lipid component, and from about 60 mole % to about 40 mole % of a first unpolymerizable lipid component.

4. The pharmaceutical composition of claim 3, wherein the composition comprises from about 45 mole % to about 55 mole % of a first polymerizable lipid component, and from about 55 mole % to about 45 mole % of a first unpolymerizable lipid component.

5. The pharmaceutical composition of claim 1, wherein greater than about 60% of the contained active ingredient is released from the population of liposomes in an environmental temperature of from about 42° C. to about 45° C.

6. The pharmaceutical composition of claim 1, wherein the first polymerizable lipid component comprises: (a) a hydrophilic head group selected from the group consisting of diethylenetriamine pentaacetic acid, ethylenedinitrile tetraacetic acid, tetraazacylododecane 1,4,7,10-tetraacetic acid, cyclohexane-1,2,-diamino-N,N-diacetatecholesterol, and a combination of two or more thereof; and (b) a hydrophobic tail group comprising a polymerizable functional group selected from the group consisting of diacetylene, olefin, acetylene nitrile, styrene, ester, thiol, amide, α-unsaturated ketone, β-unsaturated ketone, α-unsaturated aldehyde, β-unsaturated aldehyde, and a combination of two or more thereof.

7. The pharmaceutical composition of claim 1, wherein the liposome further optionally comprises cholesterol, phosphatidylcholine, phosphatidylglycerol, phosphotidylethanolamine, dioleoyphosphatidylethanolamine, distearoylphosphatidyl-choline, dioleoyphosphatidylglycerol, or a combination thereof.

8. The pharmaceutical composition of claim 1, wherein the first polymerizable lipid component comprises 23:2 1,2-bis(10,12-tricosadiynoyl)-sn-glycero-3-phosphoethanolamine or 23:2 1,2-bis(10,12-tricosadiynoyl)-sn-glycero-3-phosphocholine, or a combination thereof.

9. The pharmaceutical composition of claim 1, wherein the first polymerizable lipid component comprises 23:2 1,2-bis(10,12-tricosadiynoyl)-sn-glycero-3-phosphoethanolamine or 23:2 1,2-bis(10,12-tricosadiynoyl)-sn-glycero-3-phosphocholine, or a combination thereof.

10. The pharmaceutical composition of claim 1, wherein the first unpolymerizable lipid component comprises 22:0 1-stearoyl-2-hydroxy-sn-glycero-3-phosphoethanolamine, 20:0 1-stearoyl-2-hydroxy-sn-glycero-3-phosphoethanolamine, 18:0 1-stearoyl-2-hydroxy-sn-glycero-3-phosphoethanolamine, 16:0 1-stearoyl-2-hydroxy-sn-glycero-3-phosphoethanolamine, 14:0 1-stearoyl-2-hydroxy-sn-glycero-3-phosphoethanolamine, 12:0 1-stearoyl-2-hydroxy-sn-glycero-3-phosphoethanolamine, or 10:0 1-stearoyl-2-hydroxy-sn-glycero-3-phosphoethanolamine, or a combination thereof.

11. The pharmaceutical composition of claim 1, wherein the first unpolymerizable lipid component comprises 22:0 1,2-bis(10,12-tricosadiynoyl)-sn-glycero-3-phosphocholine, 20:0 1,2-bis(10,12-tricosadiynoyl)-sn-glycero-3-phosphocholine, 18:0 1,2-bis(10,12-tricosadiynoyl)-sn-glycero-3-phosphocholine, 16:0 1,2-bis(10,12-tricosadiynoyl)-sn-glycero-3-phosphocholine, 14:0 1,2-bis(10,12-tricosadiynoyl)-sn-glycero-3-phosphocholine. 12:0 1,2-bis(10,12 tricosadiynoyl)-sn-glycero-3-phosphocholine or 10:0 1,2-bis(10,12-tricosadiynoyl)-sn-glycero-3-phosphocholine, or a combination thereof.

12. The pharmaceutical composition of claim 1, wherein
(a) the first polymerizable lipid component comprises approximately 40 to 60 mol % of 23:2 1,2-bis(10,12-tricosadiynoyl)-sn-glycero-3-phosphoethanolamine or 23:2 1,2-bis(10,12-tricosadiynoyl)-sn-glycero-3-phosphocholine, or a combination thereof; and
(b) the first unpolymerizable lipid component comprises approximately 60 to 40 mol % of 22:0 1-stearoyl-2-hydroxy-sn-glycero-3-phosphoethanolamine, 20:0 1-stearoyl-2-hydroxy-sn-glycero-3-phosphoethanolamine, 18:0 1-stearoyl-2-hydroxy-sn-glycero-3-phosphoethanolamine, 16:0 1-stearoyl-2-hydroxy-sn-glycero-3-phosphoethanolamine, 14:0 1-stearoyl-2-hydroxy-sn-glycero-3-phosphoethanolamine, 12:0 1-stearoyl-2-hydroxy-sn-glycero-3-phosphoethanolamine, 10:0 1-stearoyl-2-hydroxy-sn-glycero-3-phosphoethanolamine, 22:0 1,2-bis(10,12-tricosadiynoyl)-sn-glycero-3-phosphocholine, 20:0 1,2-bis(10,12-tricosadiynoyl)-sn-glycero-3-phosphocholine, 18:0 1,2-bis(10,12-tricosadiynoyl)-sn-glycero-3-phosphocholine, 16:0 1,2-bis(10,12-tricosadiynoyl)-sn-glycero-3-phosphocholine, 14:0 1,2-bis(10,12-tricosadiynoyl)-sn-glycero-3-phosphocholine. 12:0 1,2-bis(10,12-tricosadiynoyl)-sn-glycero-3-phosphocholine or 10:0 1,2-bis(10,12-tricosadiynoyl)-sn-glycero-3-phosphocholine, or a combination thereof.

13. The pharmaceutical composition of claim 1, wherein the first polymerizable lipid component comprises approximately 20 to 40 mol % of 23:2 1,2-bis(10,12-tricosadiynoyl)-sn-glycero-3-phosphoethanolamine or 23:2 1,2-bis(10,12-tricosadiynoyl)-sn-glycero-3-phosphocholine, or a combination thereof; and the first unpolymerizable lipid component comprises approximately 80 to 60 mol % of 18:0 1-stearoyl-2-hydroxy-sn-glycero-3-phosphoethanolamine or 14:0 1,2-bis(10,12-tricosadiynoyl)-sn-glycero-3-phosphocholine, or a combination thereof.

14. The pharmaceutical composition of claim 1, wherein the liposomes are adapted and configured to release at least a first portion of the contained active ingredient(s) therefrom by application of heat, ultrasound, high-intensity focused ultrasound (HIFU), laser energy, photoacoustic energy, ultrasonography, light energy, or a magnetic field, or a combination thereof.

15. The pharmaceutical composition of claim 1, wherein the contained active ingredient(s) are entrapped within an interior portion of the liposomes.

16. The pharmaceutical composition of claim 1, wherein the contained active ingredient(s) are associated within or about the lipid bilayer membrane of the liposomes.

17. The pharmaceutical composition of claim 1, wherein the active ingredient(s) comprise one or more of an antineoplastic agent, an immunomodulating agent, a neuroactive agent, an antiflammatory agent, an antilipidemic agent, a hormone, a receptor agonist or antagonist, or an antiinfective agent, or a compound selected from a protein, a peptide an antibody, an enzyme, an RNA, a DNA, an siRNA, an mRNA, a ribozyme, a hormone, a cofactor, a steroid, an antisense molecule, a detection agent, an imaging agent, a contrast agent, a gas, a pharmaceutically-active molecule, and a combination thereof.

18. The pharmaceutical composition of claim 1, wherein the liposomes are stable at a pH of from about to about, preferably at a pH of from about 4.5 to 7.5, more preferably at a pH of from about 5 to about 7.

19. The pharmaceutical composition of claim 1, wherein the liposomes further comprise a first detectable, fluorogenic, radioactive, chemiluminescent, or photoluminescent label, or a combination thereof.

20. The pharmaceutical composition of claim 1, wherein at least about 50 percent, of the contained active ingredient is contained substantially within an interior portion of the liposomes.

21. The pharmaceutical composition of claim 1, wherein at least a first portion of the liposomes further comprises at least a first neutral lipid.

22. The pharmaceutical composition of claim 21, wherein the first neutral lipid comprises one or more of a cephalin, a ceramide, a cerebroside, cholesterol, diacylglycerol, diacylphosphatidylcholine, diacylphosphatidylethanolamine, phosphatidylcholine, phosphatidylethanolamine, a sphingolipid, a sphingomyelin, a tetraether lipid, or a combination thereof.

23. The pharmaceutical composition of claim 1, wherein the majority of liposomes in the population have an average diameter of from about 10 nm to about 10 μm.

24. The pharmaceutical composition of claim 23, wherein the majority of liposomes in the population have an average diameter of about 50 nm to about 5 μm.

25. The pharmaceutical composition of claim 24, wherein the majority of liposomes in the population have an average diameter of about 100 nm to about 1000 nm.

26. The pharmaceutical composition of claim 1, comprising:
(a) about 40 mole % to about 70 mole % of a first polymerizable lipid component selected from the group consisting of 23:2 1,2-bis(10,12-tricosadiynoyl)-sn-glycero-3-phosphoethanolamine and 23:2 1,2-bis(10,12-tricosadiynoyl)-sn-glycero-3-phosphocholine; and
(b) about 60 mole % to about 30 mole % of a first unpolymerizable lipid component selected from the group consisting of 22:0 1-stearoyl-2-hydroxy-sn-glycero-3-phosphoethanolamine, 20:0 1-stearoyl-2-hydroxy-sn-glycero-3-phosphoethanolamine, 18:0 1-stearoyl-2-hydroxy-sn-glycero-3-phosphoethanolamine, 16:0 1-stearoyl-2-hydroxy-sn-glycero-3-phosphoethanolamine, 14:0 1-stearoyl-2-hydroxy-sn-glycero-3-phosphoethanolamine, 12:0 1-stearoyl-2-hydroxy-sn-glycero-3-phosphoethanolamine, 10:0 1-stearoyl-2-hydroxy-sn-glycero-3-phosphoethanolamine, 22:0 1,2-bis(10,12-tricosadiynoyl)-sn-glycero-3-phosphocholine, 20:0 1,2-bis(10,12-tricosadiynoyl)-sn-glycero-3-phosphocholine, 18:0 1,2-bis(10,12-tricosadiynoyl)-sn-glycero-3-phosphocholine, 16:0 1,2-bis(10,12-tricosadiynoyl)-sn-glycero-3-phosphocholine, 14:0 1,2-bis(10,12-tricosadiynoyl)-sn-glycero-3-phosphocholine. 12:0 1,2-bis(10,12-tricosadiynoyl)-sn-glycero-3-phosphocholine, and 10:0 1,2-bis(10,12-tricosadiynoyl)-sn-glycero-3-phosphocholine,
wherein greater than about 50% of the contained active ingredient is released from the population of liposomes when the liposomes are maintained at a temperature of from about 42° C. to about 45° C. for about 5 to about 30 minutes.

27. The pharmaceutical composition of claim 1, further comprising a surfactant, a niosome, an ethosome, a transferosome, a phospholipid, a sphingosome, or a combination thereof.

28. The pharmaceutical composition of claim 1, further comprising a pharmaceutically-acceptable buffer, diluent, vehicle, or a combination thereof.

29. The pharmaceutical composition of claim 1, formulated for administration to an animal host cell.

30. The pharmaceutical composition of claim 1, formulated for administration to a human host cell.

31-39. (canceled)

40. A method for providing a therapeutic or diagnostic compound to a first cell in an animal, the method comprising, providing to the animal a therapeutically or diagnostically effective amount of a composition comprising a population of liposomes and one or more active ingredient(s) contained substantially within the population of liposomes,
wherein the composition comprises from about 30 mole % to about 70 mole % of a first polymerizable lipid component, and from about 70 mole % to about 30 mole % of a first unpolymerizable lipid component, and wherein greater than about 40% of the contained active ingredient(s) is released from the population of liposomes at a temperature of from about 39° C. to about 45° C.

41. A method for providing a prophylactic compound to an animal, the method comprising, providing to the animal at least a first prophylactically-effective amount of a composition comprising a population of liposomes and one or more active ingredient(s) contained substantially within the population of liposomes,
wherein the composition comprises from about 30 mole % to about 70 mole % of a first polymerizable lipid component, and from about 70 mole % to about 30 mole % of a first unpolymerizable lipid component, and wherein greater than about 40% of the contained active ingredient(s) is released from the population of liposomes at a temperature of from about 39° C. to about 45° C.

42. A method for providing a diagnostic or imaging agent to a selected tissue or population of target cells within the body of a mammal, the method comprising, providing to the mammal an effective amount of a composition comprising a population of liposomes and one or more active ingredient(s) contained substantially within the population of liposomes,
wherein the composition comprises from about 30 mole % to about 70 mole % of a first polymerizable lipid component, and from about 70 mole % to about 30 mole % of a first unpolymerizable lipid component, and wherein greater than about 40% of the contained active ingredient(s) is released from the population of liposomes at a temperature of from about 39° C. to about 45° C., under a condition effective to release the diagnostic or imaging agent substantially only in the target cells or tissue.

43. The method of claim 42, wherein the condition comprises providing thermal or ultrasound energy to the target cells or tissue in an amount effective to release at least 50% of the contained diagnostic or imaging agent from the liposomes present in the composition into the cells or tissue.

44. The method of claim 41, wherein the condition comprises providing thermal or ultrasound energy to the target cells or tissue in an amount effective to release at least 75% of the contained diagnostic or imaging agent from the liposomes present in the composition into the cells or tissue.

45. The method of claim 42, wherein the providing comprises photoablation, photothermal, photoacoustic, ultrasound, high intensity focused ultrasound, or laser therapy or a combination thereof.

46. The method of claim 42, wherein the diagnostic or imaging agent is detected in the selected tissue or population of target cells using computer-assisted tomography (CT) ultrasonography, magnetic resonance imaging (MRI), or a combination thereof.

47. The method of claim 42, wherein the mammal is human.

48. The method of claim 42, wherein the mammal has, is suspected of having, or at risk for developing cancer, diabetes, a neurological disorder, or a cardiovascular disease.
